(19) United States
(12) Patent Application Publication
(10) Pub. No.: US 2024/0423690 A1
(43) Pub. Date: Dec. 26, 2024

(54) FLEXIBLE CRYOTHERAPY DEVICE

(71) Applicant: Vessi Medical Ltd., Mobile Post Misgav (IL)

(72) Inventors: Eyal KOCHAVI, Haifa (IL); Mordechai SIMCHON, Haifa (IL); Koby LULEKO, Eshchar (IL)

(73) Assignee: Vessi Medical Ltd., Mobile Post Misgav (IL)

(21) Appl. No.: 18/682,973

(22) PCT Filed: Aug. 10, 2022
(86) PCT No.: PCT/IL2022/050876

§ 371 Date: Feb. 12, 2024

Related U.S. Application Data
(60) Provisional application No. 63/232,221, filed on Aug. 12, 2021.

(51) Int. Cl. A61B 18/02; A61B 1/005; A61B 1/015; A61B 1/018; A61B 1/12; A61B 18/00

(52) U.S. Cl. CPC A61B 18/0218; A61B 1/005; A61B 1/015; A61B 1/018; A61B 1/126; A61B 2018/00577; A61B 2018/0212

(57) ABSTRACT

A cryotherapy system, including:
an endoscope including:
an elongated insertion tube having a distal tip shaped and sized to penetrate into a hollow organ;
a working channel within the elongated insertion tube including a proximal opening and a distal opening at the distal tip;

a cryocatheter including:
at least one cryofluid channel with at least one distal opening positioned to release cryofluid into the hollow organ;
at least one washing fluid channel with at least one washing fluid distal opening;
wherein the cryocatheter is shaped and sized to controllably move within the working channel and to extend at least partly from the working channel distal opening, to position the at least one washing fluid distal opening in a selected distance and/or orientation relative to the elongated insertion tube distal tip.

### RELATED APPLICATION/S

This application claims the benefit of priority under 35 USC § 119 (e) of U.S. Provisional Patent Application No. 63/232,221 filed 12 Aug. 2021, the contents of which are incorporated herein by reference in their entirety.

### FIELD AND BACKGROUND OF THE INVENTION

The present invention, in some embodiments thereof, relates to a cryotherapy device and, more particularly, but not exclusively, to a flexible cryotherapy device.

### SUMMARY OF THE INVENTION

Some examples of some embodiments of the invention are listed below:

Example 1. A cryotherapy device, comprising:

an endoscope comprising:

an elongated insertion tube having a distal tip shaped and sized to penetrate into a hollow organ;

a working channel within said elongated insertion tube comprising a proximal opening and a distal opening at said distal tip;

a cryocatheter comprising:

at least one cryofluid channel with at least one distal opening positioned to release cryofluid into said hollow organ;

at least one washing fluid channel with at least one washing fluid distal opening of said at least one washing fluid channel;

wherein said cryocatheter is shaped and sized to controllably move within the working channel and to extend at least partly from said working channel distal opening, to position said at least one washing fluid distal opening in a selected distance and/or orientation relative to said elongated insertion tube distal tip.

Example 2. A device according to example 1, wherein said endoscope comprises at least one optical element at said distal tip, and wherein said cryocatheter controllably moves within said working channel to position said at least one washing fluid distal opening in a selected distance and/or orientation relative to said optical element.

Example 3. A device according to example 1, wherein said endoscope comprises at least one optical element at said distal tip defining a field of view (FOV) distal to said at least one optical element and wherein said cryocatheter controllably moves within said working channel to position said at least one washing fluid distal opening in a selected distance and/or orientation relative to said FOV.

Example 4. A device according to any one of the previous examples, wherein said at least one washing fluid channel comprises an outer wall, and wherein said at least one washing fluid distal opening comprises a plurality of washing fluid distal openings axially and/or circumferentially distributed in said outer wall.

Example 5. A device according to example 4, wherein said cryocatheter controllably extends out from the working channel according to an axial and/or circumferential distribution of said plurality of distal washing fluid openings in said outer wall.

Example 6. A device according to any one of examples 4 or 5, wherein said plurality of distal washing fluid openings are distributed in said outer wall in an area surrounding the at least one washing fluid channel.

Example 7. A device according to any one of examples 4 or 5, wherein said plurality of distal washing fluid openings are distributed in said outer wall in an area shaped as an arc partially surrounding said at least one washing fluid channel.

Example 8. A device according to any one of the previous examples, wherein said endoscope and/or said cryocatheter comprise at least one lock, configured to lock said cryocatheter at a specific position and/or at a specific orientation relative to said distal tip of said elongated insertion tube.

Example 9. A device according to example 8, wherein said lock comprises an interference lock.

Example 10. A device according to any one of the previous examples, wherein said endoscope and/or said cryocatheter comprise at least one stopper configured to limit a maximal axial extension distance of said cryocatheter from said working channel distal opening.

Example 11. A device according to any one of the previous examples, wherein said cryocatheter and/or said endoscope comprises one or more visual markings indicating a position and/or an orientation of the cryocatheter within the working channel.

Example 12. A device according to any one of the previous examples, wherein said at least one cryofluid channel is coaxially positioned within at least one washing fluid channel.

Example 13. A device according to any one of the previous examples, wherein said cryocatheter comprises a distal tip, and wherein said at least one cryofluid channel distal opening is located at said distal tip, and wherein said at least one washing fluid channel comprises at least one additional washing fluid opening in said distal tip.

Example 14. A device according to example 13, wherein said washing fluid opening in said distal tip at least partly surrounds said at least one cryofluid channel distal opening.

Example 15. A device according to any one of the previous examples, wherein a maximal inner diameter of said working channel is 4 mm.

Example 16. A device according to any one of the previous examples, comprising:

a hollow split connector comprising at least three openings, wherein an end of said hollow split connector is configured to be functionally coupled to said working channel proximal opening, and to form a flow path between said working channel and a first opening of said at least three openings, and wherein a second opening of said at least three openings comprises a connector having a seal, configured to allow insertion of said cryocatheter through said split connector into said working channel while preventing fluid release from said split connector through said connector.

Example 17. A device according to example 16, wherein said end of said split connector configured to be functionally coupled to said proximal opening is tapered.

Example 18. A device according to example 17, wherein said working channel comprises a one-way valve in said working channel proximal opening, and wherein said tapered end of said split connector is shaped and sized to penetrate and open said one-way valve.

Example 19. A device according to any one of examples 16 to 18 wherein said split connector is a Y split connector.

Example 20. A device according to any one of examples 16 to 19 wherein said seal comprises a leaf seal.

Example 21. A device according to any one of examples 16 to 20, comprising at least one sensing channel between said cryocatheter and a wall of said working channel having at least one distal opening at said distal tip, and wherein a third opening of said at least three openings of said split connector is a proximal opening of said at least one sensing channel.

Example 22. A device according to example 21, wherein said at least one sensing channel comprises a temperature or a pressure sensing channel.

Example 23. A device according to any one of the previous examples, comprising: an overtube having a tubular body with a distal end shaped and sized to penetrate into a hollow organ, wherein said overtube surrounds said endoscope and defines at least one evacuation flow path between said endoscope and an inner surface of said tubular body positioned over the endoscope to evacuate fluids from the hollow organ, wherein said at least one evacuation flow path comprises at least one distal inlet opening at said tubular body distal end, and at least one proximal outlet opening.

Example 24. A device according to example 23, wherein said overtube is selectably slidable over said endoscope.

Example 25. A device according to any one of examples 23 or 24, wherein said endoscope is coaxially positioned within said overtube tubular body.

Example 26. A device according to any one of examples 23 to 25, wherein said overtube distal end is tapered, and wherein said at least one distal inlet opening is located at a surface of said tapered distal end.

Example 27. A device according to any one of examples 23 to 25, wherein said overtube distal end is tapered, and wherein said at least one distal inlet opening is located at a surface of a non-tapered region of the overtube proximally to said tapered distal end.

Example 28. A device according to any one of examples 23 to 27, comprising an evacuation flow regulator coupled to said overtube tubular body between said at least one distal inlet opening and said at least one proximal opening of said overtube tubular body, wherein said evacuation flow regulator comprises:

at least one outlet opening configured to evacuate fluids from said overtube;

and wherein said overtube comprises a seal between said evacuation flow regulator and said at least one proximal opening of said overtube tubular body, wherein said seal is shaped and sized to seal a gap between said overtube and said endoscope in said at least one evacuation flow path to prevent passage of fluids out from said at least one proximal opening of said overtube tubular body. Example 29. A device according to example 28, wherein said evacuation flow regulator comprises at least one additional outlet opening, and a movable flow path selector configured to direct fluid flow from said at least one distal inlet opening of said evacuation flow path towards said at least one outlet opening and/or towards said at least one additional outlet opening of the evacuation flow regulator.

Example 30. A device according to example 29, wherein at least one opening of said at least one outlet opening or said at least one additional outlet opening of said evacuation flow regulator comprises a check valve configured to allow passive evacuation of fluids from said evacuation flow path.

Example 31. A device according to any one of examples 29 or 30. wherein at least one opening of said at least one first outlet opening or said at least one additional outlet opening of said evacuation flow regulator, comprises a connector configured to functionally connect a vacuum pump to said evacuation flow regulator.

Example 32. A device according to any one of examples 23 to 31, comprising:

at least one sensing channel having a distal end, wherein said at least one sensing channel travels along said elongated insertion tube between said overtube and said endoscope and wherein said at least one sensing channel distal end is coupled to said insertion tube distal tip located distally to said overtube.

Example 33. A device according to example 32, comprising a distal tip holder coupled to said elongated insertion tube distal tip, and wherein said distal tip holder is configured to interconnect said at least one sensing channel distal end to said elongated insertion tube distal tip.

Example 34. A device according to example 33, wherein said distal tip holder comprises at least one opening or at least one socket shaped and sized to receive said sensing tube distal end.

Example 35. A device according to any one of examples 33 or 34, wherein said distal tip holder at least partially surrounds said elongated insertion tube distal tip.

Example 36. A device according to any one of examples 32 to 35, wherein said at least one sensing channel comprises a temperature sensing channel or a pressure sensing channel.

Example 37. A device according to any one of examples 32 to 36, wherein a maximal diameter of said working channel is in a range between 1 to 2 mm.

Example 38. A device according to any one of the previous examples, wherein said cryocatheter comprises a distal section configured to extend out from said working channel distal opening, and wherein said device comprises at least one temperature sensing surface in said cryocatheter distal section for measuring temperature within a hollow organ.

Example 39. A device according to any one of the previous examples, comprising at least one temperature sensing surface at least partly surrounding an external surface of said cryocatheter.

Example 40. A device according to any one of the previous examples, comprising at least one temperature sensor within said elongated insertion tube of said endoscope.

Example 41. A device according to any one of the previous examples, wherein said at least one cryofluid channel in said cryocatheter comprises a distal bendable portion configured to bend in at least 45 degrees relative to a longitudinal axis of a portion of said at least one cryofluid channel located proximal to said distal bendable portion, without crimping.

Example 42. A device according to example 41, wherein said distal bendable portion of said at least one cryofluid channel has an outer diameter which is smaller than an outer diameter of said proximal portion of said at least one cryofluid channel.

Example 43. A device according to example 42, wherein said outer diameter of said distal bendable portion is in a range of 0.1-0.5 mm.

Example 44. A device according to any one of the previous examples, wherein a maximal diameter of said cryotherapy device is 10 mm.

Example 45. A cryocatheter, comprising:

an elongated body terminating with a bendable distal section, which is shaped and sized to penetrate into a hollow organ, wherein said bendable distal section comprises a distal tip;

at least one cryofluid channel located within said bendable distal section, comprises at least one distal opening at said distal tip configured to release cryofluid from said at least one cryofluid channel;

at least one washing fluid channel located within said bendable distal section, wherein said washing fluid channel comprises at least one distal opening at said distal tip configured to release washing fluid from said at least one washing channel;

wherein bending of said bendable distal section in an angle of at least 45 degrees relative to a section of the elongated body proximal to said bendable distal section, reduces cryofluid flow through said distal opening by less than 10 percent relative to cryofluid flow through said distal opening when said bendable distal section is unbent.

Example 46. A cryocatheter according to example 45, wherein a bending radius of said bendable distal section is in a range of 5-20 mm.

Example 47. A cryocatheter according to any one of examples 45 or 46, wherein said at least one cryofluid channel comprises a bendable distal section having an outer diameter smaller than 0.6 mm.

Example 48. A cryocatheter according to example 47, wherein said bendable distal section is configured to bend in at least 45 degrees without crimping.

Example 49. A cryocatheter according to any one of examples 45 to 48, wherein said elongated body has an outer diameter in a range of 1 mm to 3 mm.

Example 50. A cryocatheter according to any one of examples 45 to 49 wherein said at least one cryofluid channel is coaxially positioned within said at least one washing fluid channel in said bendable distal section.

Example 51. A working channel split connector, comprising:

a hollow body comprising at least three openings, wherein an end of said hollow body is configured to be coupled to a proximal opening of an endoscope working channel, and to form a flow path between said proximal opening and a first opening of said at least three openings, and wherein said hollow body comprises a connector at a second opening of said at least three openings, wherein said connector comprises a seal, shaped and sized to allow passage of a cryocatheter through said split connector into said working channel while preventing fluid release from said split connector through said connector.

Example 52. A connector according to example 51, wherein said end of said split connector is a tapered end shaped and sized to penetrate into said working channel proximal opening.

Example 53. A connector according to any one of examples 51 or 52 wherein said split connector is a Y split connector.

Example 54. A connector according to any one of examples 51 to 53 wherein said seal comprises a leaf seal.

Example 55. An overtube, comprising:

an elongated tubular body, comprising at least one distal opening and at least one proximal opening, wherein said elongated tubular body defines an inner lumen and is shaped and sized to penetrate at least partly into a hollow organ;

at least one flow regulator fluidically connected to said inner lumen, wherein said at least one flow regulator comprises a hollow body with at least one first outlet opening;

a seal in said elongated tubular body between said at least one flow regulator and said at least one proximal opening of said elongated tubular body, wherein said seal is configured to prevent evacuation of fluids from said elongated tubular body inner lumen through said at least one proximal opening.

Example 56. An overtube according to claim 55, wherein said at least one flow regulator comprises at least one second outlet opening; and a movable flow path selector configured to direct fluid flow from said inner lumen towards said at least one first outlet opening and/or said at least one second outlet opening.

Example 57. An overtube according to any one of examples 55 or 56, wherein an outer diameter of said overtube is smaller than 9 mm.

Example 58. An overtube according to any one of examples 56 or 57, wherein said elongated tubular body comprises a distal end shaped and sized to penetrate into said hollow organ, and wherein said at least one distal opening is located in a wall of said elongated tubular body at said distal end.

Example 59. An overtube according to example 58, wherein said at least one distal opening comprises a plurality of openings axially and/or circumferentially distributed in said elongated tubular body wall at said distal end.

Example 60. An overtube according to any one of examples 58 or 59, wherein said elongated tubular body distal end is tapered.

Example 61. An overtube according to any one of examples 56 to 60, wherein said elongated tubular body is flexible.

Example 62. An overtube according to any one of examples 56 to 61, wherein an outer surface of said elongated tubular body is smooth.

Example 63. An overtube according to any one of examples 55 to 62, wherein said at least one first outlet comprises a check valve.

Example 64. An overtube according to any one of examples 55 to 63, wherein an outer diameter of said overtube is in a range between 6 mm to 9 mm, and wherein an inner diameter of said overtube is in a range between 5 mm to 7.5 mm.

Example 65. An overtube according to any one of examples 55 to 64, wherein at least a portion of said tubular body is flexible and is configured to bend in at least 90 degrees without crimping or kinking.

Example 66. An overtube, comprising:

an elongated tubular body having a wall defining an inner lumen, wherein said inner lumen comprises at least one distal opening and at least one proximal opening, wherein said elongated tubular body is shaped and sized to penetrate at least partly into a hollow organ, wherein an inner diameter of said tubular body inner lumen is in a range between 5.5 mm and 7.5 mm;

at least one flow regulator fluidically connected to said inner lumen, wherein said at least one flow regulator comprises at least one opening;

wherein at least a portion of said elongated tubular body is configured to bend in at least 45 degrees without crimping or kinking.

Example 67. An overtube, according to example 66, wherein an outer diameter of said elongated tubular body is in a range between 6 mm and 9 mm.

Example 68. An overtube according to any one of examples 66 or 67, wherein said elongated tubular body wall is formed from metal and is coated with at least one coating layer contacting an outer surface and/or inner surface of said elongated tubular body wall, wherein said at least one coating layer is configured to prevent leakage of material from said inner volume through said elongated tubular body wall.

Example 69. A cryotherapy method, comprising:

navigating a distal section of a flexible endoscope having a working channel and a distal tip into a hollow organ;

introducing a cryocatheter having a distal end into said hollow organ via said working channel of the flexible endoscope;

controllably extending said cryocatheter distal end out from said working channel into said hollow organ to a target distance relative to said distal tip; releasing cryofluid from said cryocatheter distal end into said hollow organ;

directing washing fluid from said cryocatheter outwardly into said hollow organ before, during and/or after said releasing.

Example 70. A method according to example 69, wherein said directing comprises directing said washing fluid outwardly towards at least one optical element at said endoscope distal tip and/or towards a field of view (FOV) in said hollow organ distal to said at least one optical element.

Example 71. A method according to any one of examples 69 or 70, comprising:

bending in said hollow organ said flexible endoscope distal section in at least 45 degrees relative to a longitudinal axis of a portion of the flexible endoscope located outside the hollow organ, and wherein said releasing comprises releasing said cryofluid from said cryocatheter distal end when said flexible endoscope is bent.

Example 72. A method according to any one of examples 69 to 71, wherein said directing comprises directing washing fluid from said cryocatheter outwardly into said hollow organ through a plurality of distal washing fluid openings axially and/or circumferentially distributed in an outer wall of said cryocatheter.

Example 73. A method according to any one of examples 69 to 72, comprising locking a position and/or orientation of said cryocatheter relative to said flexible endoscope distal tip after said controllably extending.

Example 74. A cryotherapy method, comprising:

navigating a cryotherapy device comprising an endoscope and a cryocatheter into a hollow organ, wherein said cryocatheter comprises at least one cryofluid channel and at least one washing fluid channel;

bending a bendable distal section of said endoscope positioned within said hollow organ in at least 45 degrees relative to a section of said endoscope located outside said hollow organ;

releasing cryofluid from at least one distal opening of said at least one cryofluid channel located at said bendable distal section, when said bendable distal section is bent.

Example 75. A method according to example 74, comprising:

releasing washing fluid from a least one washing fluid opening of said at least one washing fluid channel located at said bendable distal section, when said bendable distal section is bent, wherein said washing fluid is released before, during and/or after the cryofluid release.

Example 76. A method according to any one of examples 74 or 75, wherein bending of said endoscope bendable distal section reduces flow of said cryofluid from said distal opening during said cryofluid releasing by less than 10 percent relative to cryofluid flow through said distal opening when said endoscope bendable distal section is unbent.

Example 77. A method for generating a passage into a hollow organ, comprising: advancing a flexible sheath having an inner lumen with a width in a range between 5 mm and 8 mm, within anatomical body lumens towards a hollow organ of a body;

bending at least a portion of said flexible sheath in at least 45 degrees during said advancing; introducing a distal end of said flexible sheath having at least one distal opening of said inner lumen into said hollow organ.

Example 78. A method according to claim 77, wherein said bending comprises bending said at least a portion of said flexible sheath while reducing a width of a cross-section of said inner lumen of said at least a portion in less than 10 percent relative to a width of said cross section of said inner lumen when said at least a portion is unbent. Example 79. A method according to any one of claim 77 or 78, comprising: draining said hollow organ and/or introducing fluid into said hollow organ via said flexible sheath inner lumen.

Example 80. A method according to any one of claims 77 to 79, comprising:

inserting a tool via said flexible sheath inner lumen into said hollow organ; and removing particles larger than 4 mm from within said hollow organ using said tool via said flexible sheath inner lumen.

Example 81. A method according to any one of claims 77 to 80, wherein said advancing comprising advancing said flexible sheath formed from a metal wall coated with at least one layer of coating, within said anatomical body lumen, wherein said at least one layer of coating seals content of said inner volume from body tissue surrounding said flexible sheath during said bending.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flow chart describing a process for using a flexible cryotherapy device, according to some exemplary embodiments of the invention;

FIG. 2A is a block diagram of a cryotherapy device which includes a working channel, according to some exemplary embodiments of the invention;

FIG. 2B is a block diagram of the cryotherapy device of FIG. 2A, with a cryocatheter positioned within the working channel, according to some exemplary embodiments of the invention;

FIG. 2C is a flow chart of a detailed process for using a flexible cryotherapy device, according to some exemplary embodiments of the invention;

FIGS. 3A-3C are schematic illustrations of a cryotherapy device, according to some exemplary embodiments of the invention;

FIG. 3D is a schematic illustration of an endoscope of a cryotherapy device, according to some exemplary embodiments of the invention;

FIGS. 4A-4C are schematic illustrations of an introducer, for example an endoscope, configuration when navigating into a hollow organ, according to some exemplary embodiments of the invention;

FIGS. 5A-5B are schematic illustrations of a cryogenic fluid catheter, for example a cryocatheter, according to some exemplary embodiments of the invention;

FIGS. 6A-6B are schematic illustrations of a cryofluid channel of a cryocatheter, according to some exemplary embodiments of the invention;

FIGS. 7A-7B are schematic illustrations showing flow of washing fluid and cryogenic fluid from a cryocatheter, according to some exemplary embodiments of the invention;

FIGS. 8A-8D are schematic illustrations showing a cryocatheter movement stopper, for example a cryocatheter movement lock of a cryotherapy device, according to some exemplary embodiments of the invention;

FIGS. 9A-9B are schematic illustrations of a washing fluid flow director, according to some exemplary embodiments of the invention;

FIGS. 10A-10D are schematic illustrations of different temperature sensors, according to some exemplary embodiments of the invention;

FIGS. 11A-11E are schematic illustrations showing an evacuation process and/or one or more evacuation flow paths of a cryotherapy device, according to some exemplary embodiments of the invention;

FIGS. 12A-12C are schematic illustrations of an interface between an overtube and an endoscope, according to some exemplary embodiments of the invention;

FIGS. 13A-13D are schematic illustrations of a split connector, according to some exemplary embodiments of the invention;

FIGS. 14A-14D are schematic illustrations of a cryotherapy device which includes at least one sensing channel located outside a working channel, according to some exemplary embodiments of the invention;

FIGS. 15A-15C are schematic illustrations of an overtube, according to some exemplary embodiments of the invention;

FIG. 15D is a schematic cross section along line A-A of the overtube shown in FIG. 15A, according to some exemplary embodiments of the invention;

FIG. 15E is a schematic cross section of an overtube having a non-circular shape, according to some exemplary embodiments of the invention;

FIG. 15F is a schematic illustration of an overtube having several sections, each with different bending capacity, according to some exemplary embodiments of the invention;

FIGS. 16A-16B are schematic illustrations showing advancing of an overtube into a hollow organ, according to some exemplary embodiments of the invention;

FIGS. 16C-16D are schematic illustrations showing flushing of an inner lumen of the hollow organ (16C), and draining of the hollow organ (16D), according to some exemplary embodiments of the invention; and

FIGS. 16E-16H are schematic illustrations showing removal of objects from the inner lumen of the hollow organ using a tool moving within the overtube, according to some exemplary embodiments of the invention.

### DESCRIPTION OF SPECIFIC EMBODIMENTS OF THE INVENTION

The present invention, in some embodiments thereof, relates to a cryotherapy device and, more particularly, but not exclusively, to a flexible cryotherapy device.

An aspect of some embodiments relates to a flexible cryotherapy device having an elongated body with a bendable distal section, comprising a cryofluid channel and a washing fluid channel in said elongated body, each having at least one opening in said bendable distal section. In some embodiments, the bendable distal section is configured to bend in at least 90 degrees, at least 150 degrees, at least 180 degrees or any intermediate, smaller or larger angle, relative to a longitudinal axis of at least one section proximal to the bendable distal section. As used herein, the term proximal means closer to an end of the cryotherapy device located outside a body of a subject, for example to a handle of the cryotherapy device, and the term distal means closer to an end of the cryotherapy device positioned within a hollow organ in the subject body, or closer to the hollow organ. In some embodiments, the flexible cryotherapy device has a maximal outer diameter of up to 10 mm, for example up to 8 mm, up to 7 mm, up to 6 mm, up to 4 mm or any intermediate, smaller or larger value. In some embodiments, the bendable distal section is configured to bend within a hollow organ, for example a hollow organ having at least one natural opening. In some embodiments, the hollow organ comprises bladder, renal pelvis, uterus, stomach or abdomen. In some embodiments, the flexible cryotherapy device is used for treating hollow organ diseases. In some embodiments, the hollow organ diseases comprise bladder cancer, interstitial cystitis, overactive bladder, gastric superficial neoplastic lesions, superficial gastric carcinoma, and/or abdominal wall tumors.

According to some exemplary embodiments, the flexible cryotherapy device optionally comprises an elongated body, for example an insertion tube, terminating with a distal bendable section. In some embodiments, a maximal width of the cryofluid channel within the distal bendable section is smaller than a maximal width of the cryofluid channel within other regions of the elongated body. In some embodiments, an outer width for example outer diameter of the cryofluid channel within the distal bendable section is in a range of 0.1-0.5 mm, for example 0.1 -0.3 mm, 0.2-0.5 mm, 0.3-0.5 mm, or any intermediate, smaller or larger range of values. In some embodiments, an outer width for example outer diameter of the cryofluid channel within at least one section of the insertion tube located proximal to the bendable distal section is in a range of 0.3-1.5 mm, for example 0.3-0.5 mm, 0.5-1 mm, 0.7-1.2 mm, 0.8-1.5 mm or any intermediate, smaller or larger range of values.

An aspect of some embodiments relates to releasing washing fluid, for example at a distal end of an introducer, for example an endoscope, outwardly towards a periphery of the endoscope. In some embodiments, the washing fluid is released from at least one washing channel, for example at least one washing tube located within a working channel of the endoscope. In some embodiments, the endoscope is a flexible endoscope, for example a cystoscope. As used herein, the term introducer refers to an introducing element which includes at least one working channel, that is configured to navigate within a body of a subject towards a desired target, for example into a hollow organ. In some embodiments, an endoscope is an example of an introducer.

According to some embodiments, the washing fluid is released towards at least one optical element of the endoscope, optionally integrated in the endoscope. In some embodiments, the at least one optical element is positioned at a distal end of the endoscope. In some embodiments, the at least one optical element comprises at least one of a lens, a window, an optic sensor, a camera, an optic fiber end, at least one light emitting diode (LED), at least one optical sensor configured to sense light with a wavelength outside the visible spectrum, and/or within the visible spectrum. Optionally, the at least one optical element comprises an optical element of a narrow band imaging (NBI) system.

According to some embodiments, the washing fluid is released from at least one opening directly towards the at least one optical element. In some embodiments, the washing fluid released towards the optic clement, for example to push away mist, dirt, and/or particles from the optical clement and/or from a field of view (FOV) of the optical element.

A potential advantage of directing washing fluid outwardly from a washing tube within the working channel of the catheter may be to avoid using any external adapters for providing washing fluid, that may block the FOV and/or that may retain mist, dirt, and/or particles in the vicinity of the optic element. Another potential advantage may be that directing washing fluid from a central region of the device towards a periphery of the device may be pushing away from the device particles, and liquid drops.

An aspect of some embodiments relates to a cryogenic fluid releasing catheter, for example a cryocatheter, that is movable within a working channel of an endoscope, for example a cystoscope, or a ureteroscope. In some embodiments, the cryofluid catheter comprises a cryofluid channel and a washing fluid channel. Optionally, the washing fluid channel comprises one or more openings configured to deliver, for example spray, washing fluid towards at least one optical element of the endoscope.

According to some embodiments, the cryofluid catheter is configured to extend out from a distal opening of the working channel, optionally into the hollow organ. Alternatively or additionally, the cryofluid catheter is pulled out from the working channel, for example to allow insertion of other surgical tools into the hollow organ.

According to some embodiments, the cryofluid catheter and/or the endoscope comprise at least one lock, for example an interference lock, configured to lock the cryofluid catheter in a specific axial, for example longitudinal position and/or in a specific angular position relative to the endoscope. In some embodiments, the at least one lock, optionally locks the cryofluid catheter in a specific position relative to a FOV and/or relative to the at least one optical element of the endoscope. Optionally, locking the cryofluid catheter in the specific position, locks one or more of the washing fluid channel openings in an axial and/or angular position which is suitable for directing washing fluid towards the FOV and/or the at least one optical element.

Potential advantages of having a movable cryofluid catheter within a working channel of a cystoscope may include (1) allowing front wash closer to the cryo fluid flow, for example to prevent or reduce cryo catheter tip frosting, (2) a simple connection to a unit that controls washing fluid flow and cryofluid flow by a single connection to the cryo catheter, (3) using of the working channel before and/or after cryofluid release for other functions, for example for taking biopsy and/or for using the working channel for evacuation of fluids.

Additional potential advantages may include retraction of a tip of the cryocatheter catheter tip during steering and/or maneuvering of the endoscope within the body, for example to reduce potential harm to tissue between treatment sessions.

According to some embodiments, the cryocatheter is configured to be introduced into a hollow organ via a working channel of an endoscope, for example, to release cryogenic fluid, for example cryofluid, and washing fluid within the hollow organ. One of the problems when having a movable cryocatheter is how to ensure a correct or optimal position of the washing fluid openings relative to at least one optical element of the endoscope, and/or relative to a field of view (FOV) between the at least one optical element and a wall of the hollow organ.

In some embodiments, one of the solutions is to have washing fluid openings that are circumferentially and/or axially distributed on a wall of the cryocatheter, for example to allow a wide-angle release of washing fluid within the hollow organ. Alternatively or additionally, another solution is to control the movement of the cryocatheter within the hollow organ according to an axially and/or circumferentially distribution of the washing fluid openings in the cryocatheter wall. In some embodiments, controlling the movement of the cryocatheter comprises limiting a maximal extension distance of the cryocatheter from the working channel, for example according to the axially and/or circumferentially distribution of the washing fluid openings. Alternatively or additionally, controlling the movement of the cryocatheter comprises limiting a rotation of the cryocatheter within the working channel, for example according to the axially and/or circumferentially distribution of the washing fluid openings. Alternatively or additionally, controlling the movement of the cryocatheter comprises locking a position of the cryocatheter within the working channel, for example according to the axially and/or circumferentially distribution of the washing fluid openings. In some embodiments, controlling a movement of the cryocatheter within the working channel is important, for example when bending of a steering region of the endoscope results with radial and/or axial movement of the cryocatheter within the endoscope working channel.

Alternatively or additionally, another solution is to provide one or more indications, for example visual indications with regard to a position and/or orientation of the cryocatheter, for example relative to the working channel and/or the endoscope. In some embodiments, the indications comprise visual markings located on the cryocatheter and/or the endoscope, for example close or at an insertion location of the cryocatheter into the working channel. In some embodiments, the indications are located outside a body, for example at a proximal section of the endoscope and/or at a proximal section of the cryocatheter. Optionally, the one or more indications are located near or at a proximal opening of the endoscope working channel through which the cryocatheter is introduced into the working channel. In some embodiments, the one or more visual markings indicate an axial and/or radial position of the cryocatheter within the working channel and/or an axial and/or radial position of the cryocatheter within the hollow organ

An aspect of some embodiments relates to a cryocatheter with an inner flexible cryofluid channel configured to deliver cryofluid towards at least one distal opening of the cryocatheter. In some embodiments, the inner flexible cryofluid channel is bendable in at least 90 degrees, for example at least 120 degrees, at least 180 degrees or any intermediate, smaller or larger value. Optionally, the cryocatheter is shaped and sized to be positioned with a flexible endoscope, for example a cystoscope.

According to some embodiments, an inner flexible cryofluid channel comprises a flexible distal portion, which is optionally shaped and sized to be positioned within a steering region of the flexible endoscope. In some embodiments, the flexible distal portion is narrower relative to a width of at least one proximal portion of the cryofluid channel. Alternatively or additionally, the flexible distal portion is formed from at least one material that is more flexible compared to at least one proximal section of the device. In some embodiments, a length of the flexible distal portion is in a range of 20-150 mm, for example 20-50 mm, 50-100 mm, 70-150 mm or any intermediate, smaller or larger range of values. In some embodiments, a bending radius of the flexible distal section is in a range of 5-20 mm, for example 5-15 mm, 10-20 mm or any intermediate, smaller or larger range of values. In some embodiments, a bending radius of at least one section of the cryofluid channel located proximal to the flexible distal portion is in a range of 70-150 mm, for example 70-100 mm, 80-120 mm, 100-150 mm or any intermediate, smaller or larger range of values.

An aspect of some embodiments relates to an overtube, for example a sheath shaped and sized to at least partly surround a flexible endoscope, and to define a fluid evacuation lumen between the flexible endoscope and overtube. In some embodiments, the sheath is an access sheath configured to generate at least one access path, for example at least one channel, into a hollow organ. In some embodiments, the access sheath is configured to generate at least one access channel between a location outside a body and a lumen of the hollow organ. In some embodiments, the fluid evacuation lumen is defined between a surface of the overtube and a surface of the flexible endoscope. In some embodiments, a maximal outer diameter of the overtube is smaller than 9 mm, for example smaller than 8 mm, smaller than 6 mm or any intermediate, smaller or larger value. Optionally, the flexible endoscope is coaxially positioned within the overtube.

According to some embodiments, a distal opening of the fluid evacuation lumen is positioned at an axial, for example longitudinal distance of up to 15 cm, for example up to 10 cm, up to 6 cm, up to 2 cm, or any intermediate, smaller or larger distance from a distal tip of the endoscope, for example to allow evacuation of fluids from a hollow organ in which the endoscope tip is positioned. In some embodiments, the overtube and/or the endoscope comprise at least one lock, configured to lock the overtube at a specific longitudinal position relative to the distal tip of the endoscope, for example to prevent uncontrolled longitudinal movement of the overtube when the endoscope is positioned within the hollow organ.

According to some embodiments, a distal opening of the fluid evacuation lumen is positioned at a longitudinal distance of up to 10 cm, for example up to 8 cm, up to 5 cm, up to 2 cm, or any intermediate, smaller or larger distance from at least one distal opening of a cryofluid channel, optionally extending out from a working channel of the flexible endoscope. According to some embodiments, the fluid evacuation lumen defined by the overtube is used to receive at least one additional channel terminating with at least one distal opening. In some embodiments, the at least one distal opening of the additional channel is positioned outside the fluid evacuation lumen, for example close to a distal tip of the endoscope. Optionally, a distal end of the additional channel is fixed to the distal tip of the endoscope, for example by a tip fixator, for example a tip holder, attached to the endoscope distal tip.

According to some embodiments, the at least one additional channel comprises a temperature sensing channel or a pressure sensing channel. In some embodiments, the at least one additional channel travels within the fluid evacuation lumen, or in a multi-lumen which includes both evacuation and sensing channels, along the endoscope body.

According to some embodiments, the sheath is flexible, and is configured to bend in an angle of at least 45 degrees, for example in at least 90 degrees, in at least 180 degrees, in at least 270 degrees, or any intermediate, smaller or larger angle.

According to some exemplary embodiments, the inner lumen of the sheath, forming at least one channel along the sheath length, has an inner width, for example an inner diameter in a range between 5 mm and 7.5 mm, for example in a range between 5 mm and 7 mm, in a range between 6 mm and 7 mm or any intermediate, smaller or larger range of values.

According to some embodiments, at least a section or all of the sheath is flexible, for example bendable. In some embodiments, bending of said flexible section in an angle of at least 45 degrees relative to a section of the sheath proximal to the flexible section, or to an unbent section of the sheath reduces a volume, optionally a local diameter of an inner lumen of the sheath by less than 20 percent, for example less than 10 percent, less than 5 percent or any intermediate, smaller or larger percentage value relative to a volume, optionally a local diameter of a section of the inner lumen where said flexible section is unbent.

According to some embodiments, the flexible sheath is used for generating a passage into a hollow organ, for example to allow at least one of draining of a lumen of the hollow organ, removal of objects from the lumen of the hollow organ and/or for introducing fluid into the hollow organ lumen. In some embodiments, the passage is generating by advancing the flexible sheath within existing anatomical body lumens, for example within the urethra, into the bladder.

A potential advantage of a flexible sheath having a wide inner lumen may be to allow easy drainage of fluid from a hollow organ and/or removal of large objects, for example large debris, large particles, stones, blood clots and sediments from within the hollow organ via the flexible sheath inner lumen. An additional potential advantage may be to allow insertion of wide and/or large tools into the hollow organ that does not fit within a working channel of an endoscope. In some embodiments, the wide inner lumen allows to use the flexible sheath for generating a wide access path into a hollow organ for draining and/or for introducing tools into the hollow organ without damaging tissue surrounding the flexible sheath.

An aspect of some embodiments relates to an evacuation flow regulator coupled to a proximal opening of an evacuation channel of a cryotherapy device. In some embodiments, the evacuation flow regulator is configured to shift an evacuation path between two openings having different flow rates. In some embodiments, the evacuation flow regulator is coupled to an overtube and an endoscope defining at least one evacuation flow path therebetween.

According to some embodiments, the evacuation flow regulator is configured to shift an evacuation flow path between at least one first opening of the flow regulator having a low flow rate, optionally used between cryofluid releasing cycles, and at least one opening of the flow regulator having a higher flow rate used when cryofluid is released within the hollow organ. Optionally the evacuation flow regulator shifts evacuation flow between at least one opening used for passive evacuation, for example an opening that includes a check valve, and at least one opening used for active evacuation, for example by a pump.

According to some embodiments, the evacuation flow regulator shifts an evacuation path, when activating cryofluid release within the hollow organ. In some embodiments, the evacuation flow regulator shifts an evacuation path when a cryofluid activation button of the flow regulator is pressed or moved. Optionally, pressing and/or moving the activation button mechanically shift the evacuation flow path. Optionally, the evacuation flow regulator shifts the evacuation flow from a single opening to two or more openings, for example when pressure in the hollow organ raises above a predetermined value. Optionally, an electrical control changes check-vale settings according to measured pressure, for example in order to control pressure.

An aspect of some embodiments relates to a split connector of a working channel of an endoscope that allows insertion of a catheter, for example a cryocatheter into the working channel, and forming a flow path between the catheter and the working channel wall, without fluid leakage from the catheter entry site. In some embodiments, the split connector comprises an inner lumen having a first opening at an end of the split connector configured to penetrate into a check valve of the working channel, and at least two separate openings, each at a different end of the split connector.

In some embodiments, at least one opening of the at least two separate openings comprises a connector, for example a gasket, which is configured to receive a cryocatheter into the split connector and into the working channel. Optionally, the gasket comprises at least one seal that allows movement of the cryocatheter within the split connector and the working channel, without allowing fluid release through the gasket. Additionally, at least one different opening of the at least two separate openings is shaped and sized to connect to a tube configured to allow a sealed fluid flow path between the tube and a lumen of the working channel surrounding the cryocatheter.

According to some embodiments, an endoscope is a guiding tool comprising a working channel, for example a central working channel. In some embodiments, the endoscope, for example the guiding tool comprises an elongated body, for example an insertion tube, having a distal tip. In some embodiments, the elongated body of the endoscope is shaped and sized to penetrate at least partly into hollow organs, for example body lumens, optionally without damaging tissue of the body lumen. In some embodiments, the endoscope comprises a working channel positioned within and along the insertion tube, comprises a distal opening at the distal tip of the endoscope.

According to some exemplary embodiments, in an endoscope with a small diameter working channel, a width, for example, diameter of the working channel is smaller than 2 mm, for example smaller than 1.8 mm, smaller than 1.5 mm, smaller than 1.2 mm or any intermediate, smaller or larger diameter. In some embodiments, the diameter of the working channel is in a range between 1-2 mm, for example 1-1.5 mm, 1.4-2 mm, 1.3-1,7 mm or any intermediate, smaller or larger range of values.

According to some exemplary embodiments, a cryocatheter sized to move within a small diameter working channel has an outer diameter which is smaller than 1.8 mm, for example smaller than 1.6 mm, smaller than 1.5 mm, smaller than 1.3 mm or any intermediate, smaller or larger value. In some embodiments, the cryocatheter outer diameter is in a range of 1.2 mm to 1.8 mm, for example 1.2 mm to 1.6 mm, 1.5 mm to 1.8 mm, 1.4 mm to 1.7 mm or any intermediate, smaller or larger range of values. In some embodiments, the cryocatheter comprises at least one cryofluid channel and at least one washing fluid channel, optionally coupled together. Optionally, the cryofluid channel is coaxially positioned with the washing fluid channel.

According to some exemplary embodiments, the cryocatheter comprises at least one proximal stopper or a proximal lock, for example close to a handle of the endoscope, for limiting the movement of the cryocatheter within the working channel. Optionally, the at least one proximal stopper or lock limit an extension of the cryocatheter from the distal opening of the working channel, to a selected extension distance. In some embodiments, the extension distance is select according a position and/or orientation of washing fluid openings of the cryocatheter relative to the endoscope distal tip or relative to an optical element of the endoscope. In some embodiments, limiting the movement of the cryocatheter to position at least one washing fluid opening at a desired orientation and/or location allows, for example, efficient release of washing fluid towards the optic element, and/or towards a field of view (FOV) between a target region in the hollow organ and the optic element. Optionally, the FOV is located distal to said optic element.

Alternatively or additionally, the cryocatheter and/or the endoscope comprise at least one distal lock or stopper to limit a movement of the cryocatheter within the working channel.

Optionally, the cryocatheter comprises at least one proximal marking, for example a visual marking located outside a subject body, for indicating an axial position and/or rotation of the cryocatheter, for example relative to the endoscope.

According to some exemplary embodiments, the cryocatheter is configured to release washing fluid within the hollow organ. In some embodiments, the cryocatheter releases washing fluid towards the optic element, and/or towards the FOV with a flow rate of at least 0.25 liter/min, for example a flow rate of at least 0.5 liter/min, at least 1.0 lit/min, at least 4 lit/min, or any intermediate, smaller or larger flow rate.

According to some embodiments, a cryofluid channel within the cryocatheter comprises an elongated channel terminating with a distal bendable section. In some embodiments, the distal bendable section is configured to bend in at least 45 degrees relative to a proximal section of the elongated channel without crimping or collapsing the cryofluid channel. In some embodiments, the distal bendable section is configured to bend in at least 45 degrees relative to a proximal section of the elongated channel while reducing an inner width of the bent section in less than 10%, for example less than 5%, less than 3%, less than 1% or any intermediate, smaller or larger percentage value, relative to an inner depth when the bent section is unbent, or straight.

In some embodiments, the distal bendable section of the cryofluid channel is thinner relative to proximal sections or the cryofluid channel. Optionally, the distal bendable section of the cryofluid channel has a thin wall, and is optionally formed from a thin walled material, for example Nitinol, Titanium, Stainless-Steel, PEEK, Nylon or braided plastic. In some embodiments, an outer width, for example an outer diameter of the distal bendable section is smaller than 0.6 mm, for example smaller than 0.5 mm, smaller than 0.4 mm, smaller than 0.2 mm, smaller than 0.1 mm or any intermediate, smaller or larger value. In some embodiments, a wall width of the distal bendable section is in a range of 0.02 mm to 0.2 mm.

In some embodiments, the bendable distal section of the cryofluid channel is positioned within a steering region of the cryocatheter and/or within a steering region of an endoscope of the cryotherapy device.

According to some embodiments, the cryofluid channel and a washing fluid channel of the cryocatheter are coupled to each other within the steering region of the endoscope, and are optionally coaxial relative to each other and/or located together within a single multi-lumen structure, for example to form a crimp resistant structure. Alternatively or additionally, a joint structure formed by the at least one washing fluid channel and the cryofluid channel is braided. Optionally, the washing fluid channel coupled to the cryofluid channel is braided. Alternatively, the joint structure, for example a structure of the cryocatheter located within the steering region of the endoscope insertion tube is formed from one or more crimp resistant material, for example

Nitinol, Titanium, Stainless-Steel, PEEK, Polytetrafluoroethylene (PTFE), and Nylon or any braided plastic.

According to some embodiments, the bendable distal section of the cryofluid channel comprises at least one distal opening, configured to release cryofluid with less than 10% reduction, for example less than 5%, less than 3% or any intermediate, smaller or larger percentage in reduction in flow rate, when bent in at least 45 degrees relative to the cryofluid flow rate when the bendable distal section is unbent. In some embodiments, when the bendable distal section is bent in at least 45 degrees, a pressure of a cryofluid within the bendable distal section is lower in less than 10%, for example lower in less than 5%, in less than 3% or any intermediate, smaller or larger percentage value compared to a pressure level of the cryofluid in a proximal unbent section of the cryofluid channel.

According to some embodiments, the endoscope comprises a working channel with an inner width, for example an inner diameter in a range of 1.8 mm to 3.5 mm, for example in a range of 1.8 mm to 2.8 mm, in a range of 2 mm to 3 mm, in a range of 2.5 mm to 3.5 mm, or any intermediate, smaller or larger range of values. In some embodiments, the working channel is located within an elongated insertion tube of the endoscope terminating with a steerable distal end. In some embodiments, the steerable distal end is configured to bend in at least 45 degrees, for example at least 90 degrees, at least 120 degrees, at least 180 degrees or any intermediate, smaller or larger value relative to a longitudinal axis of a section of the insertion tube located proximal to the distal steerable section.

According to some exemplary embodiments, a radius of bending of the steerable distal section is in a range of 5 mm to 30 mm, for example 5 mm to 20 mm, 7 mm to 25 mm or any intermediate, smaller or larger range of values.

According to some exemplary embodiments, the working channel having an inner diameter of up to 3.5 mm is sized to receive a cryocatheter having an outer diameter smaller than 3 mm, for example smaller than 2 mm, smaller than 1.8 mm or any intermediate, smaller or larger value. In some embodiments, the cryocatheter comprises at least one cryofluid channel, and at least one washing fluid channel. Optionally, the cryocatheter comprises, for example in addition to the a least one cryofluid channel and to the at least one washing fluid channel at least one additional channel, for example, a sensing channel, and/or a flow channel, in the cryocatheter body. Optionally, the cryocatheter comprises electrical wiring or optical fibers, in the cryocatheter body. In some embodiments, the at least one sensing channel is comprises to sense temperature and/or pressure. Optionally, the catheter comprises at least one additional channel defined between the catheter body and the working channel. In some embodiments, the at least one additional channel is used for sensing temperature and/or pressure. Optionally, the at least one additional channel is used to deliver fluid into the hollow organ, for example in parallel to the cryofluid and/or washing fluid.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

### Exemplary Process for Using a Flexible Cryotherapy Device

According to some exemplary embodiments, a cryotherapy device is configured to penetrate into different hollow organs and to release cryogenic fluid, within the hollow organ. In some embodiments, the cryofluid is released, and optionally sprayed within the hollow organ as part of a treatment, for example to ablate a tissue within the hollow organ. Optionally, the tissue is a cancer tissue.

According to some exemplary embodiments, at least one distal portion of the cryotherapy device, for example at least one distal portion of a cryocatheter of the device is flexible, for example bendable, in angles larger than 45 degrees, for example larger than 90 degrees, 120 degrees, larger than 150 degrees, larger than 180 degrees or any intermediate, smaller or larger angle, relative to at least one portion of the cryocatheter located proximally to the at least one distal portion.

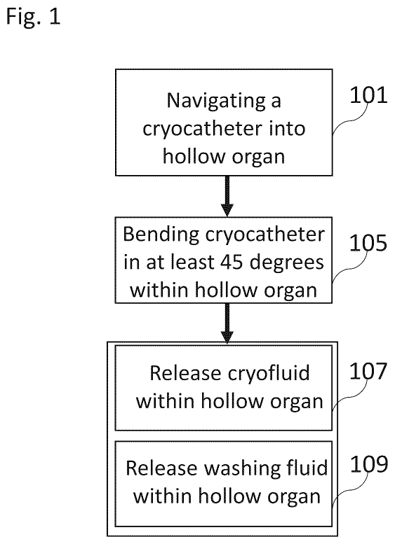

Optionally, an outer diameter of the cryocatheter is smaller than 10 mm, for example smaller than 8 mm, smaller than 6 mm, or any intermediate, smaller or larger value. Reference is now made to FIG. 1, depicting a process for navigating a flexible cryocatheter into a hollow organ, according to some exemplary embodiments of the invention; According to some exemplary embodiments, a flexible cryocatheter is navigated into a hollow organ at block 101. In some embodiments, the flexible cryocatheter comprises a bendable distal section, configured to bend in at least 90 degrees, for example at least 120 degrees, at least 150 degrees or any intermediate, smaller or larger angle. In some embodiments, the flexible cryocatheter comprises at least one cryofluid channel and at least one washing fluid channel, each has at least one distal opening in said bendable distal section. Optionally, the cryocatheter comprises at least one optical element and/or at least one fluid evacuation channel.

According to some exemplary embodiments, the cryocatheter, for example the bendable distal section of the cryocatheter bends within the hollow organ, at block 105. In some embodiments, the bendable distal section bends in at least 45 degrees, relative to at least one proximal section of the cryocatheter, within the hollow organ. In some embodiments, the at least one distal section of the cryofluid channel and the washing fluid channel bends in at least 45 degrees, according to the bending of the cryocatheter at block 105.

According to some exemplary embodiments, during and/or after the penetration of the cryocatheter into the hollow organ, the hollow organ is inflated, for example to allow visualization of the hollow organ lumen, and/or visualization of the inner surface of the hollow organ wall. In some embodiments, the hollow organ is inflated with fluid from the cryocatheter. Alternatively, the hollow organ is inflated with fluid from a channel located in an endoscope working channel, for example in a channel defined between the catheter wall and the working channel wall.

According to some exemplary embodiments, cryofluid is released within the hollow organ from the at least one cryofluid channel distal opening, at block 107. In some embodiments, the cryofluid is released within the hollow organ while the cryocatheter distal section is bent in at least 45 degrees, for example when the bendable distal section of the cryocatheter is bent in at least 45 degrees. In some embodiments, bending of a cryofluid channel distal section located within the bendable distal section of the cryocatheter in at least 45 degrees reduces cryofluid flow through a distal opening of the cryofluid channel by less than 10 percent, for example less than 7 percent, less than 5 percent, less than 2 percent, relative to cryofluid flow through the distal opening when the bendable distal section is unbent.

According to some exemplary embodiments, washing fluid is released within the hollow organ from the washing fluid channel distal opening at block 109, optionally, before, during and/or after the release of the cryofluid at block 107. In some embodiments, the washing fluid is released while the cryocatheter is bent in at least 90 degrees. According to some exemplary embodiments, the cryocatheter is shaped and sized to be introduced within an endoscope, for example a flexible endoscope where at least a distal portion of the flexile endoscope bends in an angle larger than 90 degrees, for example larger than 100 degrees, larger than 120 degrees, larger than 150 degrees or any intermediate, smaller or larger angle. In some embodiments, the cryocatheter is shaped and sized to be positioned within a working channel of the endoscope. Optionally, the cryocatheter is configured to move, for example controllably move, within the working channel.

According to some exemplary embodiments, at least one of, the inflation of the hollow organ, the release of the cryofluid, and/or the washing fluid within the hollow organ, increase the pressure within the hollow organ. In some embodiments, the pressure levels within the hollow organ is in a range of 5-100 mbar, 0-60 mbar, below 20 Ombar, above 10 mbar, in a range of 0-200 mbar, for example 0-50 mbar, 5-100 mbar, 20-200 mbar or any intermediate, smaller or larger range of values. In some embodiments, a maximal pressure level within the hollow organ is in a range of 50-200 mbar, for example 50-100 mbar, 100-200 mbar, 80-200 mbar, or any intermediate, smaller or larger range of values.

### Exemplary Flexible Cryotherapy Device Structure

According to some exemplary embodiments, a cryotherapy device is a flexible device or has a flexible distal section that is configured to bend in at least 45 degrees, for example at least 90 degrees, at least 130 degrees, at least 150 degrees or any intermediate smaller or larger angle relative to at least one proximal section of the device. In some embodiments, the flexible cryotherapy device has an outer diameter that is smaller than 10 mm, for example smaller than 8 mm, smaller than 7 mm or any intermediate, smaller or larger diameter. In some embodiments, the flexible distal section of the device is a steering section which is configured to bend, for example to place a distal end of the device close to a target tissue within a hollow organ.

According to some exemplary embodiments, the flexible cryotherapy device comprises at least one distal optical element configured to allow visualization of the hollow organ, and at least one cryofluid channel configured to deliver cryogenic fluid from a cryogenic fluid source located outside the body, to a distal opening of the cryofluid channel which is shaped and sized to be placed within a hollow organ. In some embodiments, the distal opening of the cryofluid channel is located within the flexible distal section of the device, or distally to the distal section of the device. In some embodiments, the cryofluid channel, for example a cryofluid tube is a thin tube, for example to allow flexibility and bending of the cryofluid tube. Optionally, the flexible cryotherapy device comprises a washing channel configured to deliver and release washing fluid within the hollow organ and/or towards the at least one distal optical element. Optionally the washing channel and the cryofluid channel are coaxially positioned relative to each other. Optionally, the cryofluid channel is coaxially positioned within the washing fluid channel. Additionally or optionally, the flexible cryotherapy device comprises at least one evacuation channel, configured to evacuate fluids from within the hollow organ. Exemplary components of a cryotherapy device are described in International Patent Application Publication No. WO2018142411, fled on 4-Feb-2018, the contents of which is incorporated herein as a reference in its entirety.

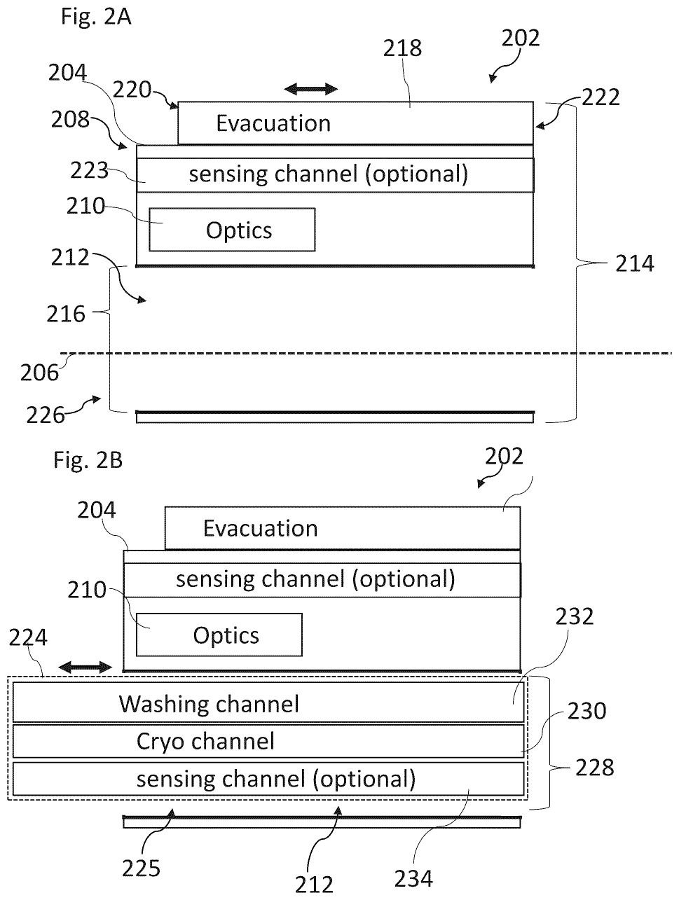

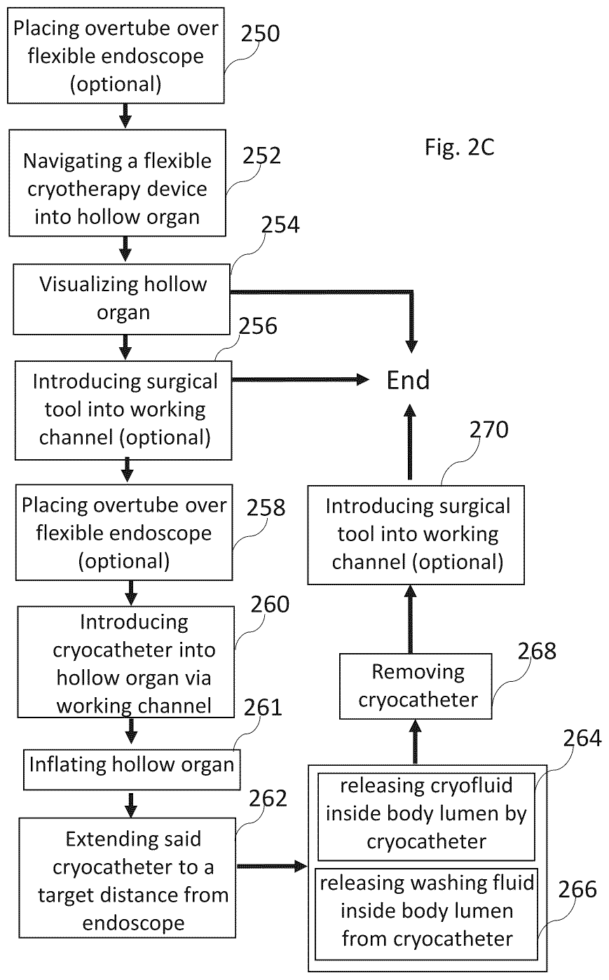

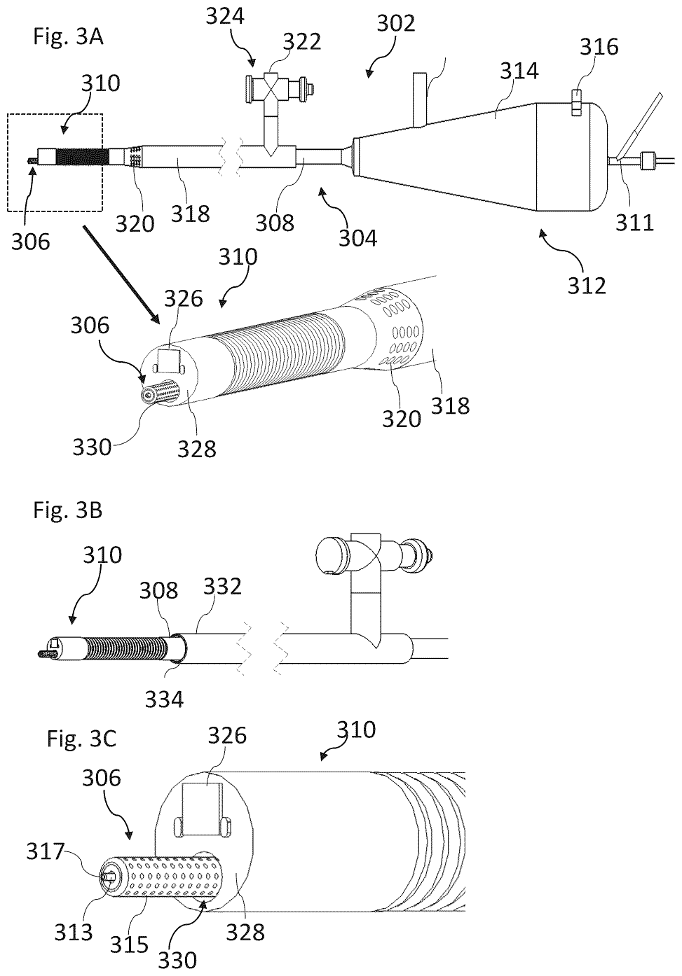

According to some exemplary embodiments, the flexible cryotherapy device comprises a flexible endoscope which includes at least one optical element, a working channel, and a cryocatheter which includes at least one cryofluid channel. In some embodiments, the cryocatheter is shaped and sized to move, for example controllably move, within the working channel. Reference is now made to FIGS. 2A and 2B, depicting a flexible cryotherapy device, according to some exemplary embodiments of the invention.

According to some exemplary embodiments, a flexible cryotherapy device 202 comprises an elongated body 204 having an elongated axis, a distal end 208, for example a steerable distal end, and a proximal end. In some embodiments, the elongated body 204 is an endoscope. In some embodiments, the elongated body 204 comprises at least one optical element in said distal end 208, for example optics 210. In some embodiments, the at least one optical element is configured to allow visualization of an area, for example a field of view (FOV) located distally to the distal end 208. In some embodiments, the at least one optical element comprises a lens, a window, an optic sensor or a camera. Additionally, the at least one optical element comprises a light emitting source, for example a lamp, a light emitting diode (LED) and/or an optic fiber end.

According to some exemplary embodiments, a maximal outer width 214 of the cryotherapy device is in a range of 1 mm-12 mm, for example 1 mm-9 mm, 1 mm-8 mm, 2 mm-8 mm, 3 mm-7 mm or any intermediate, smaller or larger range of values.

Additionally, the body 204 comprises a working channel 212 passing within the body. In some embodiments, the working channel has a maximal inner width 216 smaller than 2 mm, for example smaller than 1.8 mm, smaller than 1.6 mm, smaller than 1.4, smaller than 1.2 mm, smaller than 1 mm, or any intermediate, smaller or larger value. Optionally, an inner width 216 of the working channel is in a range of 0.2 mm-2 mm, for example in a range of 0.2 mm-1.6 mm, 0.4 mm-1.7 mm, 0.4 mm-1.5 mm or any intermediate, smaller or larger value.

Alternatively, a maximal inner width 216 of the working channel 212 is larger than 1.6mm, for example larger than 1.8 mm, larger than 2 mm, larger than 2.4 mm, larger than 3 mm, larger than 4 mm or any intermediate, smaller or larger value. Optionally an inner width 216 of the working channel is in a range of 1.6 mm-6 mm, for example in a range of 1.6 mm-5 mm, 2 mm-4 mm, 2.5 mm-5 mm or any intermediate, smaller or larger range of values. According to some exemplary embodiments, the cryotherapy device 202 comprises at least one evacuation channel 218 having a distal opening 220 located at or near the distal end 208 of the body 204. In some embodiments, the at least one evacuation channel comprises an evacuation tube located within the body 204. Alternatively, the at least one evacuation channel is formed within a lumen formed between an overtube at least partly surrounding the body 204 and the body 204, for example an external surface of the body 204. In some embodiments, the at least one evacuation channel is shaped and sized to allow evacuation, for example passive evacuation or active evacuation, of fluids from a hollow organ through the distal opening 220, and out via at least one proximal opening 222 of the at least one evacuation channel 218.

According to some exemplary embodiments, the device 202 comprises at least one sensing channel 223, within the endoscope 204. In some embodiments, the at least one sensing channel 223 is configured to sense at least one parameter, for example an environmental parameter of the hollow organ, for example humidity level, temperature, and/or pressure within the hollow organ. Optionally, the device 202 comprises at least one sensor associated, for example coupled to or located within the at least one sensing channel 223, for sensing the at least one parameter. In some embodiments, the at least one sensing channel comprises a temperature sensing channel and/or a pressure sensing channel. In some embodiments, the at least one sensor comprises a temperature sensor or a pressure sensor.

According to some exemplary embodiments, the sensing channel 223 is positioned within the elongated body 204, for example traveling within the elongated body to the distal end 208. Alternatively, the sensing channel 223 is positioned within the at least one evacuation channel 218, optionally between an overtube and the body 204. In some embodiments, the at least one sensing channel 223 is located within the body 204 and/or within the at least one evacuation channel in devices having a narrow working channel 212, for example when a maximal width 216 of the working channel 212 is smaller than 2 mm, for example smaller than 1.6 mm, smaller than 1.2 mm or any intermediate, smaller or larger value.

According to some exemplary embodiments, for example as shown in FIG. 2B, the device 202 optionally comprises a cryogenic fluid releaser, for example an elongated cryocatheter 224 within the working channel 212. In some embodiments, the cryocatheter 224 is shaped and sized to move, for example controllably move, within the working channel, from a proximal opening of the working channel, optionally located outside the body, towards a distal opening 226 of the working channel 212 at the distal end 208. In some embodiments, the cryocatheter 224 moves controllable within the working channel, manually or by using a motor, for example an electric motor, optionally operated by a user of the cryotherapy device. In some embodiments, the cryocatheter is configured to extend at least partly out from the distal opening, for example up to a distance of 10 cm, for example up to 5 cm, up to 2 cm, up to 0.5 cm or any intermediate, smaller or larger distance from the distal opening 226.

In some embodiments, the cryocatheter and/or the body 204 comprise a lock, for example a movement lock, configured to lock the cryocatheter 224 in a specific orientation and/or in a specific position, relative to the body 204. Optionally, the movement lock is configured to lock the cryocatheter 224 in a specific orientation and/or in a specific position within the working channel 212.

According to some exemplary embodiments, the cryocatheter 224 is a flexible cryocatheter or has at least one flexible distal section. In some embodiments, the cryocatheter 224 has a maximal outer width in a range of 0.5 mm to 5 mm, for example in a range of 0.5 mm to 2 mm, in a range of 1 mm to 3 mm, in a range of 3 mm to 5 mm, or any intermediate, smaller or larger range of values. In some embodiments, the flexible catheter or the at least one distal section of the catheter is configured to bend in at least 45 degrees, for example at least 90degrees, at least 120 degrees, at least 150 degrees or any intermediate, smaller or larger value, for example relative to axis 206 of the device 202 or relative to a more proximal section of the cryocatheter 224.

According to some exemplary embodiments, the cryocatheter 224 comprises at least one cryofluid releasing channel, for example cryo channel 230 disposed within the elongated cryocatheter 224. In some embodiments, the cryo channel comprises at least one proximal opening functionally connected to a cryogenic fluid source, and at least one distal opening configured to be positioned within a hollow organ and to release cryogenic fluid, also termed herein as cryo fluid into the hollow organ.

According to some exemplary embodiments, the cryocatheter 224 comprises at least one washing fluid channel, for example washing channel 232. In some embodiments, the washing channel 232 is positioned within the elongated catheter 224. In some embodiments, the washing channel comprises at least one proximal opening functionally connected to a washing fluid source, and at least one distal opening configured to release washing fluid within a hollow organ, optionally towards the at least one optic element 210. In some embodiments, the at least one washing fluid channel is formed by a tube.

According to some exemplary embodiments, the at least one washing channel 232 and the at least one cryo channel 230 are coupled to each other, for example coaxially coupled to each other. Alternatively, the at least one washing channel 232 and the at least one cryo channel 230, are located, optionally side-by-side, within the cryocatheter body. Optionally, the at least one cryo channel 230 is coaxially positioned within the at least one washing channel 232. In some embodiments, movement of the cryo catheter 224 within the working channel 212 moves the cryo channel 230 together and optionally in synchronization, with the washing channel 232.

Alternatively, the at least one washing channel 232 is configured to move independently relative to the cryo channel 230 within the cryocatheter and/or within the working channel 212. Optionally, movement of at least one of the cryo catheter 224, the at least one cryo channel 230 and/or the at least one washing channel 232 comprises axially movement and/or rotation.

Optionally, the cryo catheter 224 comprises at least one sensing channel 234, used to sense at least one parameter of the hollow organ, for example at least one environmental parameter of the hollow organ, for example as described with regard to sensing channel 224. In some embodiments, the cryo catheter 224 comprises the at least one sensing channel 234 when the working channel 212 is wide enough to receive the cryo catheter, for example when a width of the working channel is at least 1.6 mm, for example at least 1.8 mm, at least 2 mm or any intermediate, smaller or larger value. According to some exemplary embodiments, the device 202 optionally comprises at least one additional channel 225, for example a sensing channel or a fluid channel, defined between an outer wall of the cryocatheter 224 and an inner wall of the working channel 212. Optionally, the at least one additional channel 225 terminates with the working channel distal opening 226. In some embodiments, the at least one additional channel comprises a temperature sensing channel and/or a pressure sensing channel, for example as described above with respect to channels 223 or 234. In some embodiments, the device 202 optionally comprises the at least one additional channel, when an inner diameter of the working channel 212 is larger than 1.8 mm, for example larger than 2 mm, larger than 2.3 mm or any intermediate, smaller or larger value, for example to allow the presence of both the cryocatheter 224 and the at least one additional channel 225 within an inner lumen of the working channel 212.

According to some exemplary embodiments, at least one of the channels of the device 202 is formed from at least one tube. In some embodiments, the at least one tube is flexible or has a flexible region, and/or is optionally formed from a flexible material. Alternatively or additionally, at least a portion of the tube is configured to bend in at least 45 degrees, for example at least 90 degrees, at least 150 degrees or any intermediate, smaller or larger angle, relative to an unbent portion of the tube, optionally without crimping or kinking. In some embodiments, the portion of the tube is configured to bend in at least 45 degrees relative to an unbent portion of the tube while reducing an inner width of a cross-section of the bent portion in less than 10%, for example less than 5%, less than 3%, less than 1% or any intermediate, smaller or larger percentage value, relative to an inner width of the cross section when the bent portion is unbent, or straight.

According to some exemplary embodiments, the cryotherapy device 202 is functionally coupled to a control unit, for example as described in International Patent Application Publication No. WO2018142411, fled on 4-Feb-2018, the contents of which is incorporated herein as a reference in its entirety. In some embodiments, the control unit is configured to control at least one of release of cryo fluid from the at least one cryo channel 230 and/or the release of washing fluid from the at least one washing channel 232, optionally according to measurements of the at least one parameter of the hollow organ. Optionally, the at least one control unit monitors the movement, for example the steering, of the cryotherapy device 202 and/or the cryo catheter 224 towards the hollow organ and/or within the hollow organ. An exemplary control unit and/or control of a cryotherapy device or a cryo catheter is described in International Patent Application Publication number WO2018142411 filed on 4-Feb-2018, the content of which is incorporated herein as a reference in its entirety.

### Exemplary Detailed Cryotherapy Process

Reference is now made to FIG. 2C depicting a detailed cryotherapy process using a flexible cryotherapy device, according to some exemplary embodiments of the invention.

According to some exemplary embodiments, an overtube is optionally placed around an elongated insertion tube of a flexible cryotherapy device, at block 250. In some embodiments, the overtube is optionally positioned around an elongated insertion tube of an endoscope of the flexible cryotherapy device. In some embodiments, the elongated insertion tube is introduced into the inner lumen of the overtube, optionally when the elongated insertion tube is outside a body of a subject.

According to some exemplary embodiments, the flexible cryotherapy device, comprising the flexible endoscope is navigated through an opening in the body into a hollow organ, at block 252. In some embodiments, a distal section of the flexible cryotherapy device is introduced into the hollow organ via at least one opening of the hollow organ. In some embodiments, the at least one opening of the hollow organ is an anatomical opening. Alternatively, the at least one opening of the hollow organ is a surgical opening, formed by at least one surgical instrument, for example a knife, a scalpel, or scissors.

According to some exemplary embodiments, the hollow organ, for example a lumen of the hollow organ is visualized at block 254. In some embodiments, at least one optical element located at a distal tip of the cryotherapy device, for example at a distal tip of the endoscope of the flexible cryotherapy device, is used to visualize the hollow organ. In some embodiments, the distal section of the endoscope is bended, within the hollow organ, for example to position a distal opening of a working channel of the cryotherapy device, for example a working channel of the endoscope, in a target location within the hollow organ. Optionally, the endoscope is steered within the hollow organ to position the working channel distal opening in proximity, for example at a distance shorter than 5 cm, for example shorter than 2 cm, shorter than 1 cm or any intermediate, smaller or larger distance from a target tissue in the hollow organ.

Optionally, after visualizing the hollow organ, the cryotherapy device is removed from the hollow organ, for example when an expert decided that a treatment is not required.

According to some exemplary embodiments, at least one surgical tool is optionally introduced through the working channel into the hollow organ, at block 256. In some embodiments, the at least one surgical tool comprises a biopsy device, a cold-cup biopsy forceps, a coagulating device, a fulguration device. In some embodiments, the biopsy device extends out from the working channel into the hollow organ and removes a biopsy sample from a tissue within the hollow organ. Optionally, after removal of the biopsy device from the working channel, the flexible cryotherapy device, for example the flexible endoscope of the cryotherapy device is removed from the body.

Optionally, an overtube is placed around a flexible endoscope of the cryotherapy device at block 258. In some embodiments, the overtube is positioned around the insertion tube of the flexible endoscope, as described at block 250.

According to some exemplary embodiments, a cryocatheter is introduced into a working channel of the flexible endoscope, at block 260. In some embodiments, the cryocatheter moves within the working channel into the hollow organ.

According to some exemplary embodiments, the hollow organ is inflated at block 261, for example after penetration of the endoscope into the hollow organ. In some embodiments, the hollow organ is inflated, for example by releasing fluid, for example air, carbon dioxide or other type of gas into the hollow organ, from the endoscope and/or the cryocatheter into the hollow organ. Optionally, the hollow organ is inflated by releasing cryofluid and/or washing fluid from the endoscope and/or the cryocatheter into the hollow organ. In some embodiments, fluids are evacuated through at least one distal opening of the overtube during the inflation of the hollow organ, for example to maintain a pressure level within the hollow organ within a target, optionally predetermined, range of pressure levels. According to some exemplary embodiments, the cryocatheter or at least a distal section of the cryocatheter, extends out from the working channel into the hollow organ, to a target distance from the endoscope, at block 262. Optionally, the cryocatheter extends out from the working channel to a distance of up to 5 cm, for example up to 2 cm, up to 0.5 cm, from a distal tip of the endoscope. In some embodiments, the cryocatheter extends to a distance selected to position an opening of a washing fluid channel of the catheter at a target distance from an optical element of the endoscope. Optionally, the cryocatheter extends to a distance selected to position an opening of the washing fluid channel at a target orientation relative to the optical element.

According to some exemplary embodiments, cryofluid is released from at least one opening of the cryofluid channel inside the hollow organ, at block 264. In some embodiments, the cryofluid is released, optionally sprayed, towards a target tissue within the hollow organ. In some embodiments, the cryofluid is released from an opening of the cryofluid channel located at a portion of the cryocatheter extending out from the working channel. According to some exemplary embodiments, washing fluid is released inside the hollow organ from within the working channel, at block 266. In some embodiments, the washing fluid is released in a timed relation with the release of the cryofluid, for example before, during and/or after the release of the cryofluid at block 264. In some embodiments, the washing fluid is released from at least one opening of a washing fluid channel, for example a washing fluid tube, positioned within the working channel of the endoscope. In some embodiments, the washing tube extends out from the working channel into the hollow organ, and positions the at least one opening of the washing tube outside the working channel and inside the hollow organ. Optionally, the at least one opening of the washing tube is positioned and/or oriented within the hollow organ to release, optionally to direct, washing fluid towards the at least one optical element at the distal tip of the endoscope.

According to some exemplary embodiments, the washing fluid is directed outwardly towards a distal tip of the endoscope, for example towards at least one optical element at the distal tip. Alternatively or additionally, the washing fluid is directed towards the FOV and/or towards the treated area or a target region within the hollow organ.

Optionally, sensing fluid flow, is introduced into the hollow organ, before, during and/or after the release of the cryofluid and/or the washing fluid. In some embodiments, the sensing fluid flow is used to measure pressure within the hollow organ.

According to some exemplary embodiments, the cryocatheter is removed from the working channel at block 268.

According to some exemplary embodiments, at least one additional tool, for example a surgical tool, is optionally inserted into the working channel of the endoscope at block 270. Optionally, the at least one additional tool is inserted into the hollow organ via the working channel. In some embodiments, the at least one additional tool comprises a biopsy device, a cutting tool, a coagulating device, and/or fulguration device. In some embodiments, the at least one additional tool is configured to remove a tissue, for example a tissue previously ablated by the cryofluid, from the hollow organ.

### Exemplary Flexible Cryotherapy Device

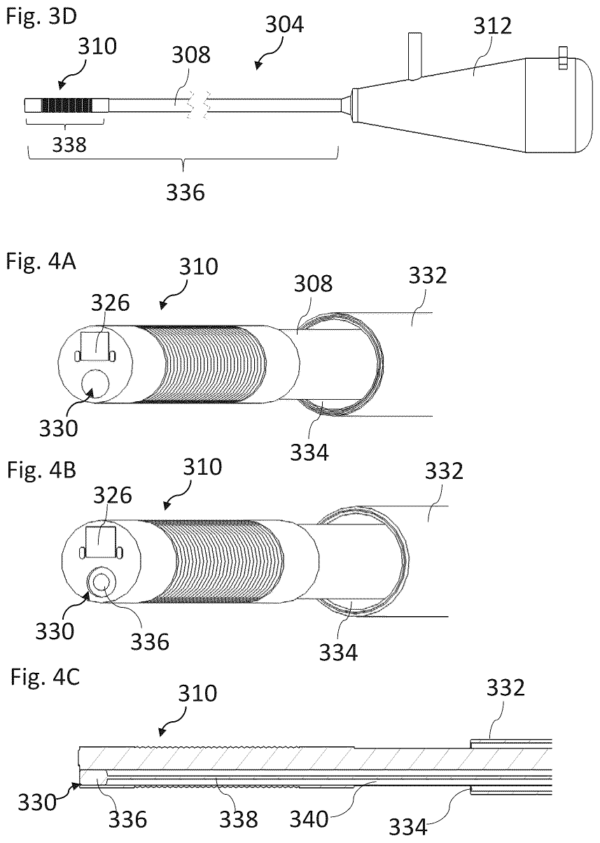

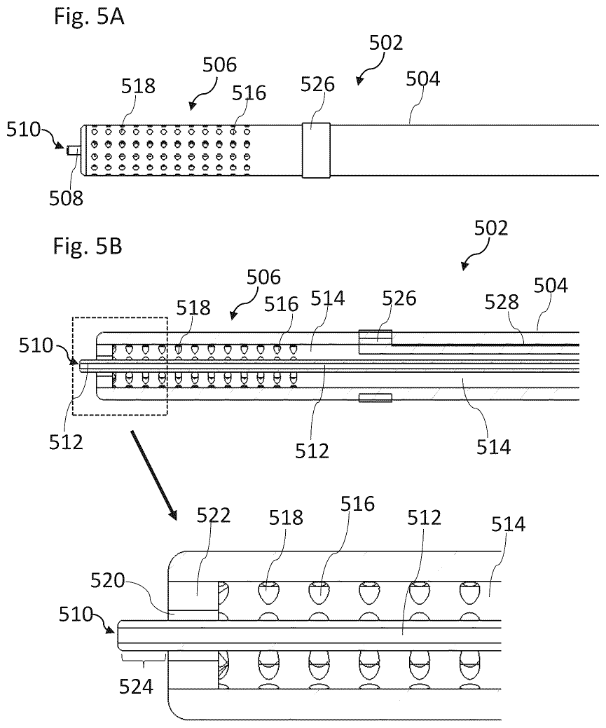

Reference is now made to FIGS. 3A-3B, depicting a flexible cryotherapy device, according to some exemplary embodiments of the invention.

According to some exemplary embodiments, a flexible cryotherapy device, for example device 302 comprises an endoscope 304, for example a cystoscope, and a cryo catheter 306 positioned, for example movably positioned, within a working channel of the endoscope 304. In some embodiments, the endoscope 304 comprises an elongated body 308, for example an elongated insertion tube, having a distal section 310 and a proximal section 312. In some embodiments, the elongated insertion tube terminates with the distal section 310, which is a steering section configured to flex and/or bend in at least 45 degrees, for example 90 degrees, 120 degrees, 150 degrees or any intermediate, smaller or larger angle.

According to some exemplary embodiments, the endoscope 304 comprises a handle 314 functionally coupled to the insertion tube 308 in the proximal section 312. In some embodiments, the handle 314 is configured to control the navigation of the insertion tube 304 within the body and towards a target hollow organ, for example by controlling the bending of the distal section 310 of the insertion tube 308. In some embodiments, the control handle 312 comprises at least one steering controller 316, functionally coupled to the insertion tube 308 and/or to the distal section 310 of the insertion tube 308. In some embodiments, the at least one steering controller is configured to control the steering of the distal section 310, for example by applying mechanical force on the insertion tube 308 and/or the distal section 310, for example by stretching cables attached to the steering section, for example the distal section 310.

According to some exemplary embodiments, the device 302 comprises at least one evacuation channel 318, for example evacuation channel having at least one distal inlet opening 320, and at least one proximal outlet opening 322. In some embodiments, the at least one evacuation channel 318 is configured to evacuate fluids, for example liquids, gases, and/or particles, from a hollow organ into which the distal section 310 of the device 302 is introduced. In some embodiments, fluids and/or particles are introduced passively or actively into the distal inlet opening 320 placed within the hollow organ, and are evacuated from the at least one evacuation channel 318 via the at least one proximal outlet opening 322. In some embodiments, passive evacuation comprises evacuation based on differences in pressure levels between the hollow organ and external environment. In some embodiments, active evacuation comprises evacuation using a vacuum pump that reduces pressure levels within the at least one evacuation channel relative to pressure levels within the hollow organ.

According to some exemplary embodiments, the device 302 comprises at least one evacuation flow regulator 324 coupled to the at least one evacuation channel 318. Optionally, the evacuation flow regulator 324 comprises the at least one proximal outlet opening 322.

According to some exemplary embodiments, the at least one evacuation channel 318, is optionally formed by a tube that at least partly or entirely surrounds the insertion tube 308. Optionally, the insertion tube 308 is coaxially disposed within the at least one evacuation channel 318. In some embodiments, a lumen of the at least one evacuation channel 318, through which the fluids and/or particles are evacuated is formed between a tube, for example an overtube forming the at least one evacuation channel, and an external surface of the insertion tube 318. In some embodiments, the at least one evacuation channel 318 is slidable, for example selectably slidable, relative to the insertion tube 308.

According to some exemplary embodiments, the at least one distal inlet opening 320 comprises a plurality of openings in the wall of the evacuation channel 318, optionally at a distal end of the evacuation channel 318. In some embodiments, the distal end is a tapered distal end, shaped and sized to fit to, and/or around the insertion tube 308. Optionally, the tapered distal end allows a graded transition between the evacuation channel and the insertion tube.

According to some exemplary embodiments, the endoscope 304 comprises at least one optical element, for example optical element 326 at a distal tip 328 of the distal section 310. In some embodiments, the optical element comprises at least one of, a window, an aperture, an optic sensor, a camera, a lens, and/or a light source.

According to some exemplary embodiments, the endoscope 304 comprises a working channel traveling within the insertion tube 308, having at least one working channel distal opening 330, and at least one working channel proximal opening. In some embodiments, the working channel is shaped and sized to receive a cryocatheter 306 that is configured to move within the working channel, and optionally to extend from the distal opening 330, for example when the distal opening 330 is positioned within the hollow organ.

According to some exemplary embodiments, for example as shown in FIG. 3B, a distal opening 334 of an evacuation channel is formed between an external surface of the insertion tube 308 and an inner surface of a tube, for example an overtube 332 forming the evacuation channel.

In some embodiments, the distal opening 334 is shaped as a ring or an arc, optionally at least partly surrounding the insertion tube 308.

According to some exemplary embodiments, the device 302 comprises at least one insertion tube splitter 311, for example a split connector coupled to the insertion tube 308, and configured to divide a proximal opening of the insertion channel into two or more openings.

According to some exemplary embodiments, for example as shown in FIG. 3C, the cryocatheter 306 comprises a cryofluid channel, for example a cryofluid tube 313 disposed, for example coaxially disposed within a washing fluid channel 315. In some embodiments, the washing fluid channel 315 comprises a plurality of openings, for example two or more washing openings, in the wall of the channel 315. In some embodiments, the washing openings are distributed on a circumference of the washing fluid channel 315, for example to allow release and/or direction of washing fluid towards the optical element 326.

According to some exemplary embodiments, the cryocatheter 306 extends out from a distal opening of the working channel 330, for example to place an opening 317 of the cryofluid channel 313 close, for example at a distance of up to 4 cm, for example up to 3 cm, up to 2 cm, up to 1 cm up to 0.5 cm or any intermediate, smaller or larger distance, from a target tissue within the hollow organ. Alternatively or additionally, the cryocatheter 306 extends out from working channel 330 to a distance that places at least one washing opening of the plurality of washing openings in a desired location and/or a desired orientation relative to the optical element 326.

Optionally, a distal opening of the cryofluid channel 313, for example opening 317 is located distally to the washing fluid channel 315. Optionally, the cryofluid channel 313, is configured to move, for example controllably move within the washing fluid channel 315. Alternatively, the cryofluid channel 313 and the washing fluid channel 315 are coupled to each other, and optionally move in synchronization or together.

Reference is now made to FIG. 3D, depicting an endoscope 304, for example a cystoscope, according to some exemplary embodiments of the invention.

According to some exemplary embodiments, the endoscope 304, as describe above comprises an elongated insertion tube 308, terminating with a distal section 310, for example a steerable section. In some embodiments, a proximal end of the insertion tube is coupled to the handle 312. In some embodiments, a length 336 of the insertion tube 308 is in a range of 20-100 cm, for example 30-70 cm. 25-60 cm, 40-80 cm or any intermediate, smaller or larger range of values. In some embodiments, a length 338 of the distal section 310, for example a steerable section, is in a range of 3-9 cm, for example 3-8 cm, 4-7 cm, 5-9 cm or any intermediate, smaller or larger range of values

### Exemplary Endoscope Insertion

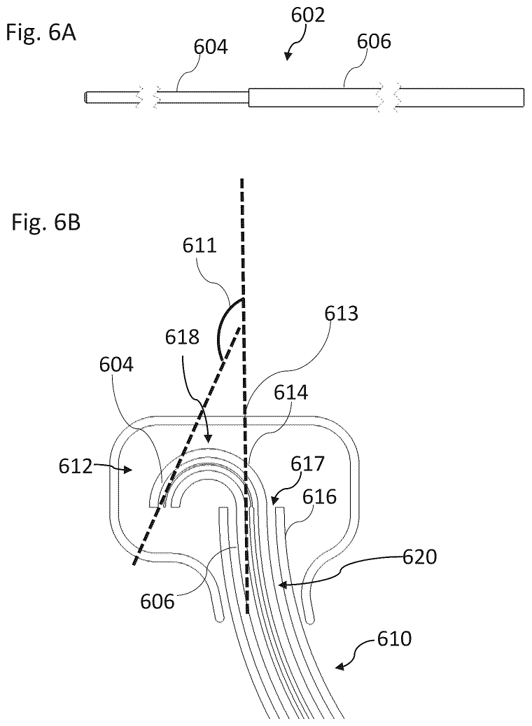

Reference is now made to FIGS. 4A to 4C depicting a configuration of an endoscope, for example a flexible cystoscope, when entering into a hollow organ, according to some exemplary embodiments of the invention.

According to some exemplary embodiments, a cryotherapy device is introduced into a hollow organ, by first navigating an endoscope into the hollow organ. In some embodiments, the endoscope comprises an insertion tube terminating with a flexible, for example bendable distal section 310. In some embodiments, the insertion tube comprises a working channel terminating with a distal opening 330.

According to some exemplary embodiments, for example as shown in FIGS. 4A to 4C, an overtube 332 positioned around the insertion tube 308 forms at least one evacuation channel between the overtube 332 and the insertion tube 308, with a distal evacuation opening 334. In some embodiments, during navigation of the endoscope and/or when the distal section 310 is positioned within the hollow organ, the overtube 332 is located proximally to the distal section 310. Optionally, the distal evacuation opening 334 is positioned within the hollow organ, for example to allow evacuation of fluids and/or particles from the hollow organ. Evacuation of fluids and/or particles from within the hollow organ may be important in order to maintain pressure levels in the hollow organ within a predetermined range of pressure levels, for example to prevent high pressure levels that may cause damage to the hollow organ tissue, and to prevent pressure levels that are too low for organ inflation, for example for navigation and/or visualization purposes inside the hollow organ.

According to some exemplary embodiments, during the navigation of the endoscope into the hollow organ the working channel opening 330 remains open. Alternatively, during the navigation process of the endoscope into the hollow organ, the opening is closed with a removable plug 336. In some embodiments, closing the opening 330 with the removable plug 336 allows, for example, to prevent entry of tissue and unwanted body material into the working channel 340 of the endoscope. In some embodiments, the removable plug 336 is coupled to a wire and/or a flexible rod 338, optionally positioned within the working channel 340. Optionally, the wire and/or flexible rod 338 is coupled to the handle 312. In some embodiments, retraction of the wire and/or the flexible rod 338, removes the plug 336 from the opening 330, to allow, for example, insertion of the cryocatheter and/or other tools via the working channel 340 into the hollow organ.

### Exemplary Cryogenic Fluid Catheter

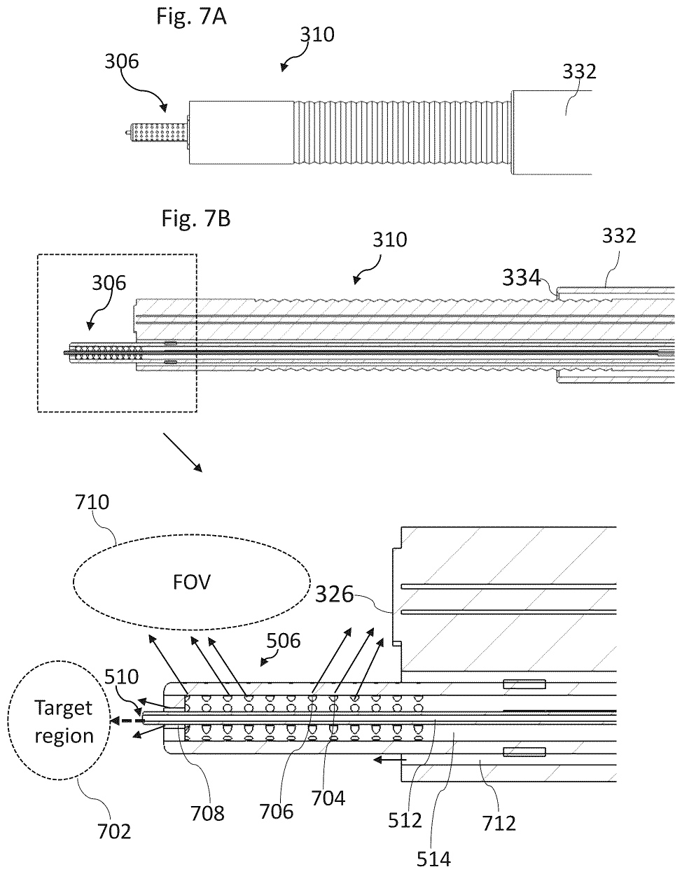

Reference is now made to FIGS. 5A and 5B, depicting a cryogenic fluid catheter, for example a cryocatheter, according to some exemplary embodiments of the invention.

According to some exemplary embodiments, a cryocatheter 502 comprises an elongated body 504 with a distal section 506. In some embodiments, the cryocatheter 502 comprises at least one cryofluid tube 508, having at least distal opening 510, configured to allow release of cryogenic fluid, for example cryofluid, from a cryofluid channel 512 within the cryofluid tube 508 or formed by the cryofluid tube 508.

According to some exemplary embodiments, for example as shown in FIGS. 5B and 5C, the cryocatheter 502 comprises at least one washing channel 514 positioned within the elongated body 504. In some embodiments, the washing channel 514 comprises one or more, for example a plurality of openings at the distal section 506 of the elongated body 504. In some embodiments, the washing channel openings are shaped, sized and/or positioned to release washing fluid from the washing channel 514 into the hollow organ. Optionally, the washing channel openings are shaped, sized and/or positioned to direct washing fluid towards at least one optical element of the endoscope, for example optical element 326 shown in FIGS. 3A-3C.

According to some exemplary embodiments, the washing fluid openings are axially and/or angularly distributed on a circumference of the washing fluid channel 514. In some embodiments, the washing fluid openings are angularly distributed to completely surround the washing channel 514. Alternatively, the washing fluid openings are angularly distributed on an arc subtending an angle of at least 2 degrees, for example 5 degrees, 10 degrees, 30 degrees, 90 degrees, 180 degrees or any intermediate, smaller or larger angle. In some embodiments, the washing fluid openings have a maximal width or a maximal diameter in a range of 0.01 mm-2 mm, for example 0.01 mm-1 mm, 0.05 mm-0.7 mm, 0.1 mm-1.5 mm or any intermediate, smaller or larger range of values.

According to some exemplary embodiments, the cryofluid channel 512 is coaxially positioned within the washing fluid channel 514. In some embodiments, the cryofluid channel is coupled, for example contacts the washing fluid channel 514 along at least 30%, for example at least 40%, at least 50%, at least 70%, at least 80% or any intermediate, smaller or larger percentage value of a length of the cryofluid channel 512. According to some exemplary embodiments, the washing channel 514 comprises at least one distal forwardly facing opening, for example opening 520, which is configured to direct washing fluid in axial alignment with cryofluid released from opening 510. In some embodiments, the cryocatheter 502 comprises at least one washing fluid flow director, for example flow director 522, optionally positioned within the washing channel 514. In some embodiments, the flow director, for example flow director 522 is positioned within the distal end of the washing channel 514, between the washing channel 514 and the cryofluid channel 512. In some embodiments, the flow director 522 comprises the opening 520 that surrounds at least partly the cryofluid channel 512 passing through the flow director 522. Optionally, the flow director is a flow regulator, configured to regulate the amount of washing fluid directed towards the optical element and the amount of washing fluid directed towards a tip of the cryofluid channel, for example towards the opening 510.

According to some exemplary embodiments, an axial, for example longitudinal distance 524 between the cryofluid channel opening 510 and the washing fluid channel opening is a fixed distance and is in a range of (−) 1-10 mm, for example (−) 1-0.1 mm, (−) 0.5-0.5 mm, 0-1 mm, 0-3 mm, 0-5 mm, 0-7 mm or any intermediate, smaller or larger value. In some embodiments, the cryofluid channel opening 510 is distal to the washing fluid channel opening. Alternatively, the cryofluid channel 512 is configured to axially move, for example controllably move, within the washing fluid channel 514, and the axial distance 524 varies.

According to some exemplary embodiments, the cryocatheter 502 comprises at least one sensor or two or more sensors, for example a temperature sensor and/or a pressure sensor. In some embodiments, the at least one sensor is coupled to the body 502 and/or is positioned within the body. Optionally, the at least one sensor is positioned within at least one of, the washing fluid channel, the cryofluid channel and/or optionally in at least one additional channel. According to some exemplary embodiments, the at least one sensor comprises a thermocouple or a temperature sensing surface, optionally shaped as an arc or as a ring. In some embodiments, the temperature sensing surface, for example temperature sensing surface 526 is coupled to an external surface of the body 504. Optionally, the surface 526 is located proximal to the washing fluid openings, for example not to interfere and/or to block a release of washing fluid through the washing fluid opening and/or not to be influenced by the washing fluid flow. Optionally, the temperature sensing surface is electrically connected by electrical wiring 528, positioned within the elongated body 504 of the cryocatheter, to a control unit.

### Exemplary Cryofluid Channel

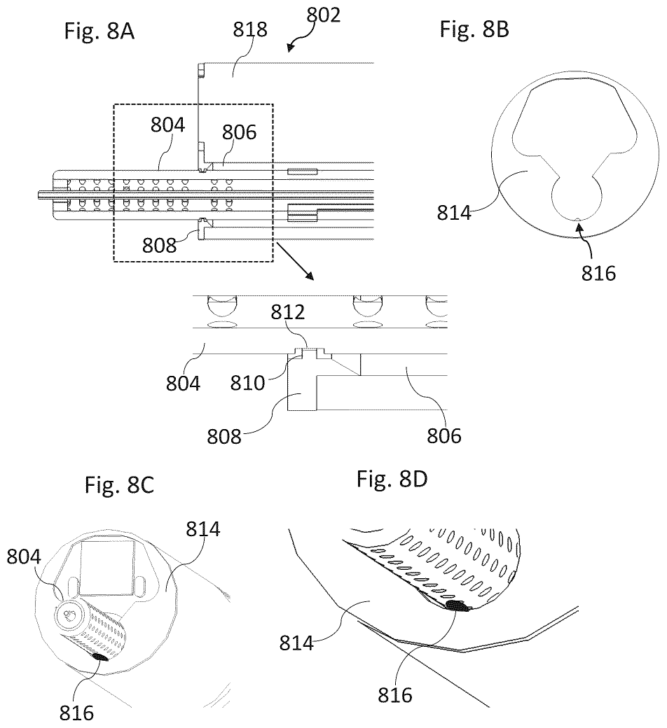

Reference is now made to FIGS. 6A and 6B, depicting a cryofluid channel, according to some exemplary embodiments of the invention.

According to some exemplary embodiments, a cryofluid channel of a cryocatheter is flexible or has at least one flexible section, for example a distal section terminating with the distal opening of the cryofluid channel. In some embodiments, for example as shown in FIG. 6A, the cryofluid channel is formed from an elongated cryofluid tube 602 comprising a distal flexible section 604. In some embodiments, the distal flexible section 604 is configured to bend in angle 611 relative to a longitudinal axis 613 of a proximal section 606. In some embodiments, the angle 611 is at least 45 degrees, for example at least 60 degrees, at least 90 degrees, at least 120 degrees, at least 150 degrees, at least 180 degrees, or any intermediate, smaller or larger angle.

According to some exemplary embodiments, the flexible section 604 is thinner than the proximal section 606, and has a width or an outer diameter in a range of 0.1-2 mm, for example 0.1-0.7 mm, 0.1-1 mm, 0.5-1.5 mm or any intermediate, smaller or larger range of values. In some embodiments, a length of the distal section 604 of the cryofluid tube 602 is in a range of 20-150 mm, for example 20-50 mm, 30-100 mm, 50-150 mm or any intermediate, smaller or larger range of values. In some embodiments, the radius of curvature of the flexible section 604 is smaller than the radius of curvature of the proximal section 606.

According to some exemplary embodiments, for example as shown in FIG. 6B, a cryotherapy device, for example device 610 is introduced into a hollow organ, for example a hollow organ 612. In some embodiments, an endoscope 614 surrounded at least partly with an overtube 616 is introduced into a hollow organ 612, optionally placing a distal opening 617 of the overtube 616 within the hollow organ 612. In some embodiments, the endoscope 614 comprises a distal steering section 618 and an insertion tube 620. Optionally, the insertion tube 620 terminates with the distal steering section. In some embodiments, the steering section 618 is configured to bend in at least 45 degrees, for example at least 60 degrees, at least 120 degrees, at least 150 degrees, at least 180 degrees or any intermediate, smaller or larger angle, relative to the insertion tube 620. In some embodiments, the steering section 618 bends when the endoscope is steered to a desired, a selected and/or a predetermined location within the hollow organ 612. Optionally, the steering section 618 bends within the hollow organ 612, for example when visualizing the interior of the hollow organ 612 using the at least one optical element of the endoscope, for example optical element 326 shown in FIGS. 3A to 3C.

According to some exemplary embodiments, a length of the steering section 618 of the endoscope 614 is in a range of 20-150 mm, for example 50-150 mm, 70-120 mm, 30-100 mm or any intermediate, smaller or larger range of values. In some embodiments, a length of the insertion tube 620 is in a range of 250-700 mm, for example 250-400 mm, 350-450 mm, 400-700 mm or any intermediate, smaller or larger range of values. In some embodiments, a length of the distal flexible section 604 of the cryofluid tube 602 is at least in a length of the steering portion 618, or is selected according to a length of the steering portion 618.

A potential advantage of positioning a thin cryotube in a steering region of the endoscope may be to allow bending of the cryotube together with the bending of the steering region while efficiently delivering cryofluid through the cryotube into the hollow organ.

### Exemplary Flow of Fluids

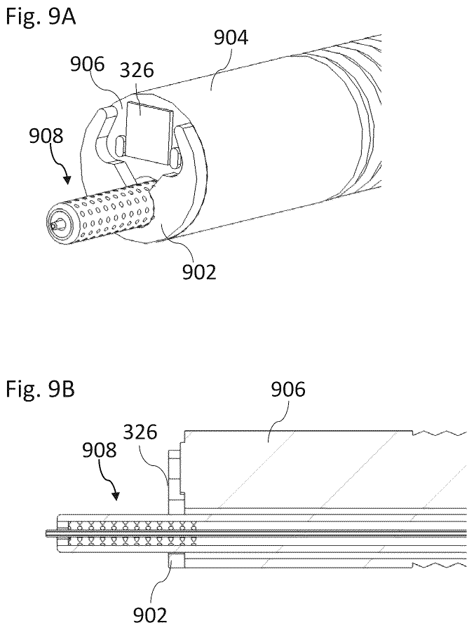

Reference is now made to FIGS. 7A-7B depicting flow of fluids to and/or from the cryotherapy device, according to some exemplary embodiments of the invention.

According to some exemplary embodiments, a distal section 306 of a cryocatheter positioned outside a working channel of an endoscope comprises at least one distal opening 510 of a cryofluid tube 512 configured to direct, for example spray cryofluid towards a target region 702 located distally to the cryocatheter. In some embodiments, a washing fluid channel 514, comprises at least one opening, for example openings 704 and 706 that are configured to direct washing fluid towards at least one optical element 326 of the endoscope. In some embodiments, the openings are positioned in a wall of the washing fluid channel 514, optionally on a circumference of the washing fluid channel. Alternatively or additionally, the washing fluid channel 514 comprises at least one forwardly facing distal opening, for example opening 708, configured to direct washing fluid towards the target region 702. Alternatively or additionally, the washing fluid channel 514 comprises at least one opening in the wall of the washing fluid channel 514, configured to direct washing fluid towards a field of view (FOV) 710, located between the at least one optical element 326 and the target region.

According to some exemplary embodiments, the release of washing fluid from the washing channel, into the hollow organ, for example towards the target region 702, towards the FOV 710 and/or towards the at least one optical element 326 is performed in synchronization and/or according to the release of cryofluid into the hollow organ, for example through the opening 510.

According to some exemplary embodiments, fluids and/or particles are evacuated through opening 334 of the overtube 332. In some embodiments, evacuation of fluids and/or particles through the opening 334 is optionally performed according to pressure levels within the hollow organ, and/or in order to reach a specific range of pressure levels within the hollow organ.

According to some exemplary embodiments, for example as shown in FIG. 7B, the cryotherapy device comprises at least one additional channel, for example a pressure sensing channel 712 located within the working channel of the endoscope. In some embodiments, the pressure sensing channel 712 is positioned between the working channel wall and the outer wall of the cryocatheter. Optionally the pressure sensing channel 712, at least partly or entirely surrounds the cryocatheter positioned within the working channel. In some embodiments, the pressure sensing mechanism uses constant flow with a constant pressure drop through the channel, and with pressure sensor at the proximal end of the channel, for example inside a control unit. Optionally, when the pressure in the hollow organ is changed, the pressure in the pressure sensor changes accordingly, in a way that the pressure drop through the channel remains constant, hence the pressure inside the hollow organ can be calculated according to the measured proximal pressure and the known constant pressure drop.

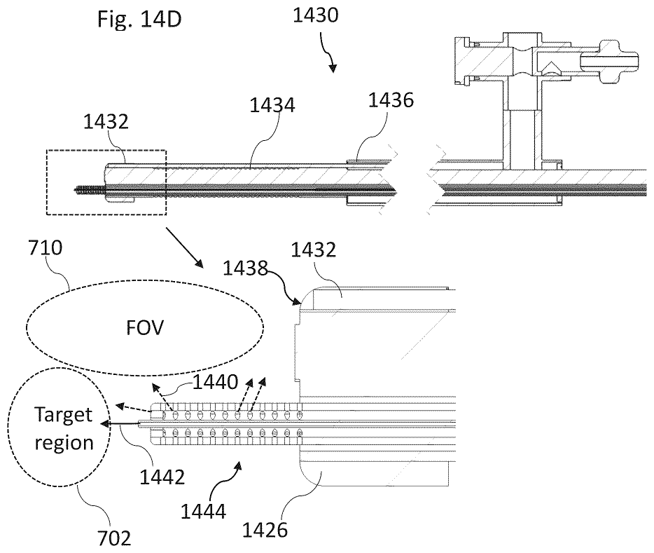

Alternatively, for example as shown in FIGS. 14A-14C, if the working channel of the endoscope is too narrow to accommodate a cryocatheter and at least one sensing channel, for example a temperature or a pressure sensing channel, the at least one sensing channel is integrated in the endoscope, or passes in a lumen formed between the overtube and the endoscope. Alternatively, the at least one sensing channel is located within an overtube that comprises multiple inner lumens.

### Exemplary Cryocatheter Stopper

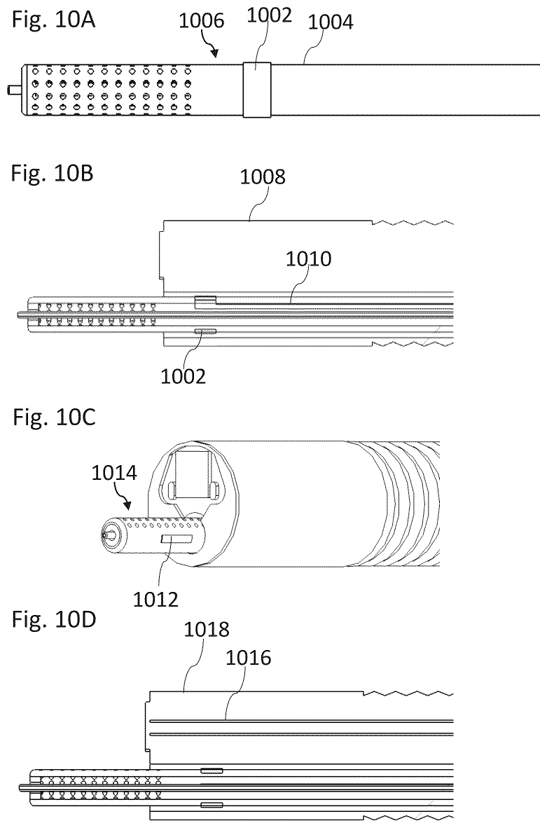

According to some exemplary embodiments, the cryocatheter is movable within the working channel of the endoscope, for example to allow advancement of the cryocatheter distally to the endoscope to apply cryofluid and/or washing fluid. Alternatively or additionally, the cryocatheter is movable within the working channel, for example to cover the cryocatheter while navigating within the body towards the hollow organ and/or to allow retraction of the cryocatheter from the working channel and insertion of at least one additional surgical tools into the hollow organ. In some embodiments, the cryotherapy device comprises a stopper, configured to limit or stop a movement, for example axial movement and/or rotation, of the cryocatheter within the working channel. Reference is now made to FIGS. 8A-8D depicting a movement stopper, for example a movement lock, according to some exemplary embodiments of the invention.

According to some exemplary embodiments, a cryotherapy device, for example device 802 comprises a cryocatheter, for example cryocatheter 804 that is configured to move within a working channel 806 of the device 802, for example a working channel of an endoscope of the device 802. Optionally, the cryocatheter 804 is retracted completely out from the working channel 806, or is not positioned within the working channel 806, for example when navigating the cryotherapy device into a target hollow organ.

According to some exemplary embodiments, for example as shown in FIG. 8A, the device 802 comprises a stopper, for example a lock 808 configured to stop or limit an axial movement of the cryocatheter 804 within the working channel 806. In some embodiments, the lock 808 is coupled to the working channel 806, and comprises at least one protrusion 810, for example a bulge, which is shaped and sized to fit into a channel 812 in the external surface of the cryocatheter. In some embodiments, the channel 812 at least partly surrounds the cryocatheter 804. Optionally, the channel 812 is shaped as a ring. Alternatively, the channel is shaped as an arc. In some embodiments, insertion of the protrusion 810 into the channel 812 locks the catheter 804 at a specific axial, for example longitudinal location within the working channel 806, while optionally allowing rotation of the catheter 804 within the working channel 806, when the protrusion 810 moves within the channel 812. In some embodiments, a degree of rotation is determined based on a length of the channel 812.

According to some exemplary embodiments, the lock is coupled to the cryocatheter 804, and a protrusion of the lock is shaped and sized to fit into a channel within the working channel 806 or within a locking element coupled to the working channel 806.

According to some exemplary embodiments, for example as shown in FIGS. 8B to 8D, the lock, for example lock 814 comprises a protrusion 816, for example a bulge. In some embodiments, the lock 814 is configured to be coupled to a distal end of the endoscope 818. In some embodiments, the protrusion 816 is shaped and sized to fit into a pointed recess or a pointed opening in the cryocatheter, for example into a washing channel opening. In some embodiments, insertion of the protrusion 816 into a pointed recess or a pointed opening, locks the cryocatheter in a specific longitudinal and rotational position within the working channel. In some embodiments, locking the cryocatheter in a specific longitudinal and rotation position allows to place specific washing fluid openings on the circumference of the cryocatheter in a desired location relative to the at least one optical element 326 and/or relative to the FOV 710, shown in FIG. 7B.

### Exemplary Washing Fluid Flow Director

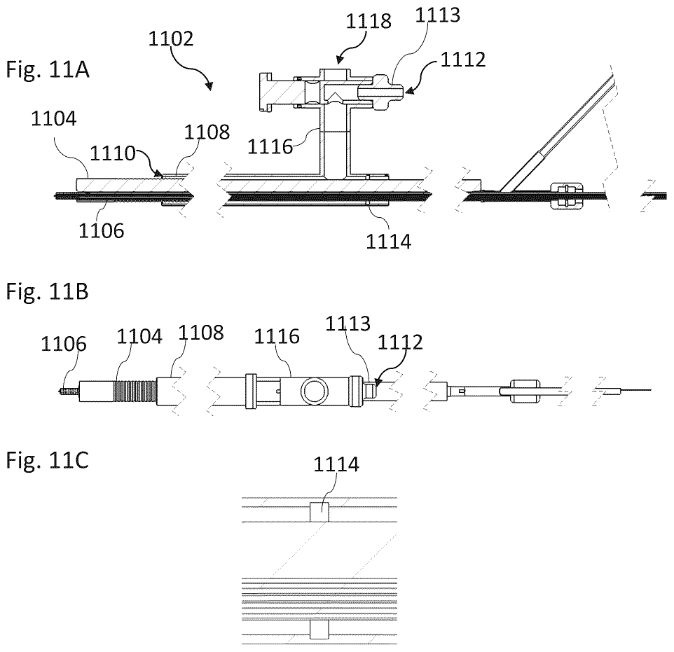

According to some exemplary embodiments, the cryotherapy device comprises at least one washing fluid flow director configured to direct washing fluid from selected washing fluid openings in the washing channel at a specific direction. Reference is now made to FIGS. 9A and 9B, depicting at least one washing fluid flow director, according to some exemplary embodiments of the invention.

According to some exemplary embodiments, the cryotherapy device comprises at least one washing fluid flow director, for example wash flow director 902 coupled to the endoscope 904. Optionally, the wash flow director 902 is coupled, for example attached to a distal end 906 of the endoscope. In some embodiments, for example as shown in FIG. 9A, the wash flow director 902 partially surrounds a distal opening of a working channel of the endoscope 904 through which a cryocatheter 908 exits.

According to some exemplary embodiments, the director 902 is configured to block washing fluid openings positioned to direct washing fluid away from the at least one optical element 326, while unblocking washing fluid openings that are positioned to direct washing fluid toward the optical element 326.

According to some exemplary embodiments, the director 902 is shaped and sized to partially surround a washing fluid channel of the cryocatheter 908. In some embodiments, the director 902 surrounds at least 25% of a circumference of a portion of the washing channel that includes washing fluid openings, for example at least 50% of the circumference, at least 75% of the circumference, at least 80% of the circumference, at least 90% of the circumference, at least 95% of the circumference, or any intermediate, smaller or larger percentage value of the circumference of the washing channel portion containing the washing openings.

Optionally, the lock 814 shown in FIGS. 8A to 8D is used as a washing fluid director.

### Exemplary Temperature Sensing

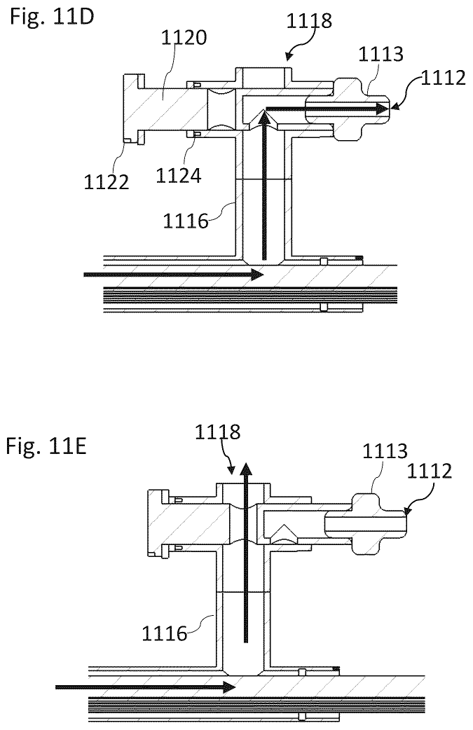

According to some exemplary embodiments, the cryotherapy device comprises at least one sensor for sensing temperature within at least one channel of the cryocatheter and/or within a hollow organ. In some embodiments, sensing temperature is important, for example not to damage tissue, and/or not to affect the optical element, within the hollow organ if the temperature is lower than a predetermined value. Reference is now made to FIGS. 10A-10D depicting temperature sensing by the cryotherapy device, according to some exemplary embodiments of the invention.

According to some exemplary embodiments, a cryotherapy device comprises at least one temperature sensor, for example a heat sensing surface 1002 coupled to the external surface of a cryocatheter 1004. In some embodiments, the heat sensing surface 1002 is coupled to a surface of the cryocatheter 1004 at a section that is configured to remain within the working channel of an endoscope 1008. In some embodiments, the heat sensing surface 1002 at least partially surrounds the cryocatheter 1004. Optionally, the heat sensing surface is shaped as a ring or as an arc. In some embodiments, the heat sensing surface 1002 is electrically connected by wires 1010 to a control unit located outside the body of the subject. In some embodiments, the wires are positioned within the cryocatheter, located inside the working channel of the endoscope 1008. Optionally, the heat sensing surface 1002 is configured to sense a temperature within the working channel, for example the temperature near the cryocatheter, optionally close to the working channel exit, for example the working channel distal opening.

According to some exemplary embodiments, for example as shown in FIG. 10C, a cryotherapy device comprises at least one heat sensing surface coupled to a distal section 1014 of a cryocatheter, for example cryocatheter, that is configured to be positioned outside the working channel of the endoscope in the hollow organ. Optionally, the sensing surface at the distal section 1014 of the cryocatheter is configured to sense temperature within the hollow organ.

According to some exemplary embodiments, for example as shown in FIG. 10D, a cryotherapy device comprises at least one sensor, for example a temperature sensor integrated in the endoscope 1018.

According to some exemplary embodiments, a cryotherapy device comprises at least one temperature located in at least one of, on a surface of the cryocatheter in a section positioned within a working channel of an endoscope of the device, on a surface of the cryocatheter in a section positioned outside of the working channel, and/or integrated or coupled to the endoscope.

Exemplary evacuation

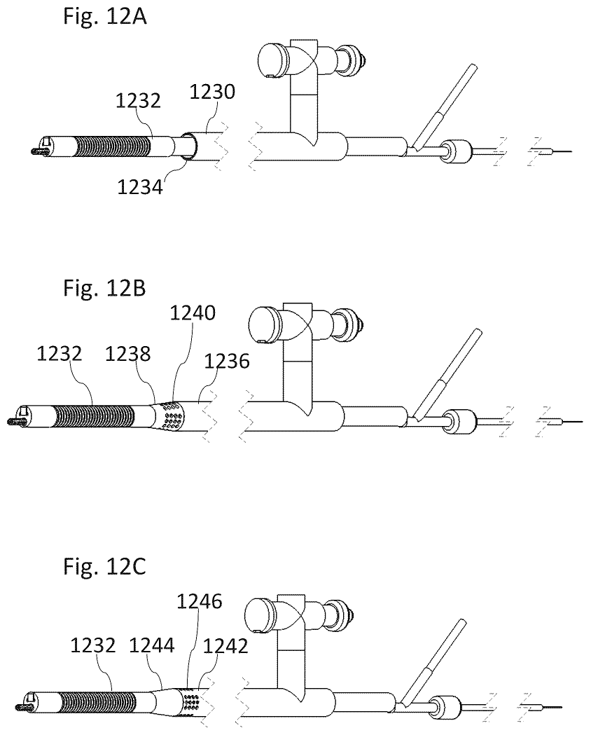

According to some exemplary embodiments, a cryotherapy device comprises at least one evacuation channel configured to evacuate fluids from a hollow organ, for example to prevent increase in pressure levels within the hollow organ that may result with tissue damage and/or tissue rupture. Reference is now made to FIGS. 11A-11C depicting a cryotherapy device with at least one evacuation channel, according to some exemplary embodiments of the invention.

According to some exemplary embodiments, a cryotherapy device 1102 comprises an endoscope 1104, a cryocatheter 1106 positioned at least partly, and optionally coaxially, within the endoscope 1104, and an outer sheath, for example overtube 1108, surrounding at least partly the endoscope 1104. In some embodiments, the overtube 1108 is selectably slidable over the endoscope, for example to control a distance between a distal opening of the overtube and a distal tip of the endoscope within the hollow organ. In some embodiments, the overtube 1108 forms at least one evacuation channel in a lumen between the overtube 1108 and the endoscope 1104. In some embodiments, the overtube 1108 comprises at least one distal inlet opening, for example distal inlet 1110 shaped and sized to receive fluids from within a hollow organ, and at least one proximal outlet opening, for example proximal outlet 1112, for releasing evacuated fluids from the evacuation channel.

According to some exemplary embodiments, the device further comprises at least one seal, for example seal 1114 positioned in the at least one evacuation channel proximally to the outlet 1112. In some embodiments, the seal 1114 is positioned between the overtube 1108 and the endoscope 1104, and is configured to seal the at least one evacuation channel proximally to the outlet 1112. Optionally, the seal 1114 is shaped as a ring. In some embodiments, the seal 1114 is formed from rubber, silicone or any other sealing material. Alternatively, the seal 1114 comprises at least one leaf seal, configured to allow relative movement and maneuverability while preventing backward leakage between the overtube 1108 and the endoscope 1104, so evacuation is channeled towards a planned exit, for example a vent element optionally comprising a check valve 1113.

According to some exemplary embodiments, for example as shown in FIGS. 11A and 11B, the device comprises at least one evacuation flow regulator 1116 fluidically connected to the at least one evacuation channel, and optionally comprises at least one outlet opening of the evacuation channel, for example outlet 1112. According to some exemplary embodiments, the evacuation flow regulator 1116 comprises one or more outlet openings of the at least one evacuation channel, for example at least one outlet opening for passive evacuation, for example outlet opening 1112, and at least one outlet opening for active evacuation, for example outlet opening 1118. In some embodiments, the outlet opening 1112 optionally comprises a check valve 1113. In some embodiments, the outlet opening 1118 optionally comprises an adaptor for connecting the evacuation flow regulator to an external evacuation device, for example to a pump.

Reference is now made to figs, 11D and 11E, depicting changes in evacuation states, according to some exemplary embodiments of the invention.

According to some exemplary embodiments, flow regulator is configured to switch between at least two outlet openings, for example outlet openings 1112 and 1118 during the activation of the cryotherapy device. In some embodiments, at least one opening, for example opening 1112 comprises a check valve 1113. According to some exemplary embodiments, between cryo activation cycles when cryofluid is released within the hollow organ, the check valve in opening 1112 is used for fluid evacuation, for example to maintain a minimal pressure level within the hollow organ. In some embodiments, for example as shown in FIG. 11E, when cryofluid is released within the hollow organ, pressure levels elevate, and the flow regulator 1116 directs flow within the evacuation channel towards outlet 1118 which is configured to allow a higher rate of flow compared to opening check valve 1113.

According to some exemplary embodiments, the flow regulator 1116 changes the evacuation flow path between the two openings based on signals received from at least one sensor, for example a pressure sensor. Optionally, the flow regulator 1116 changes the evacuation flow path between the two openings, automatically, for example using a movable flow path selector, for example a movable seal, positioned within the flow regulator 1116. In some embodiments, the flow regulator movable seal is configured to move between at least one first state where flow is directed towards opening 1112, and at least one second state, where flow is directed towards at least one different opening, for example towards opening 1118.

According to some exemplary embodiments, for example as shown in FIGS. 11D and 11E, the flow regulator 1116 comprises a cryo release activation button, for example activation button 1120. In some embodiments, movement of the activation button is configured to release cryofluid from the cryocatheter, for example within a hollow organ, and to switch an evacuation flow path from opening 1112 to opening 1118. In some embodiments, the activation button activates the cryofluid release electrically, for example when the activation button moves towards the flow regulator 1116, and an electrical contact 1122 of the button contacts an electrical contact 1124 on the flow regulator 1116. Additionally, movement of the activation button 1120 mechanically moves the movable seal. Alternatively, movement of the activation button electrically activates cryofluid release and moves the movable seal, for example by activating an electric motor.

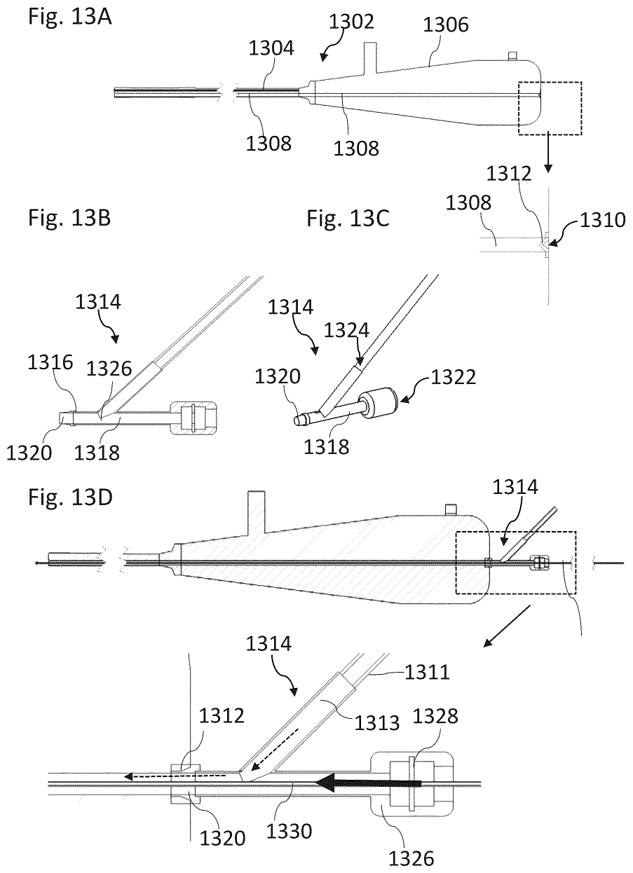

Reference is now made to FIGS. 12A-12C depicting an overtube with different distal ends, according to some exemplary embodiments of the invention.

According to some exemplary embodiments, for example as shown in FIG. 12A, an overtube 1230 surrounds the endoscope 1232 and has at least one distal opening 1234 between the endoscope 1232 and the overtube 1230. In some embodiments, the overtube 1230 and the opening 1234 has a substantially similar inner diameter, for example an inner diameter with variations in the inner diameter or in the outer diameter of the endoscope, smaller than 0.5%, for example smaller than 0.3%, smaller than 0.1%, along at least 95% of a length of the overtube 1230. Optionally, a wall of the overtube 1230 is thin, for example has a thickness in a range of 0.05 mm to 0.4 mm, for example 0.05 mm to 0.1 mm, 0.08 mm to 0.2 mm, 0.1 mm to 0.3 mm or any intermediate, smaller or larger value.

According to some exemplary embodiments, for example as shown in FIG. 12B, an overtube 1236 surrounding endoscope 1232 has a tapered distal end 1238. In some embodiments, the tapered distal end 1238 comprises at least one or a plurality of distal openings of the evacuation flow path formed between the overtube 1236 and the endoscope 1232. In some embodiments, the openings are located in the wall of the tapered distal end 1238. A potential advantage of placing evacuation openings in a tapered distal end of the cryotube may be to prevent blocking of the evacuation openings by tissue that is attached to non-tapered, straight walls of the overtube. An additional potential advantage of the tapered end may be that it allows and easier and/or smoother insertion of the overtube into the body, following the endoscope insertion.

According to some exemplary embodiments, for example as shown in FIG. 12C, an overtube 1242 comprises a tapered distal end 1244, and at least one or a plurality of distal openings 1246 of the evacuation flow path located in a wall of a straight section, of the overtube 1242. Optionally, distal openings are located in a wall of a tapered region and in a wall of a straight region of the overtube.

### Exemplary Split Connector

According to some exemplary embodiments, in order to use a working channel of an endoscope for delivering of a cryocatheter into a hollow organ, and for forming at least one channel, for example between the cryocatheter and the working channel wall, a connecter, for example a split connector, is coupled to a proximal opening of the working channel.

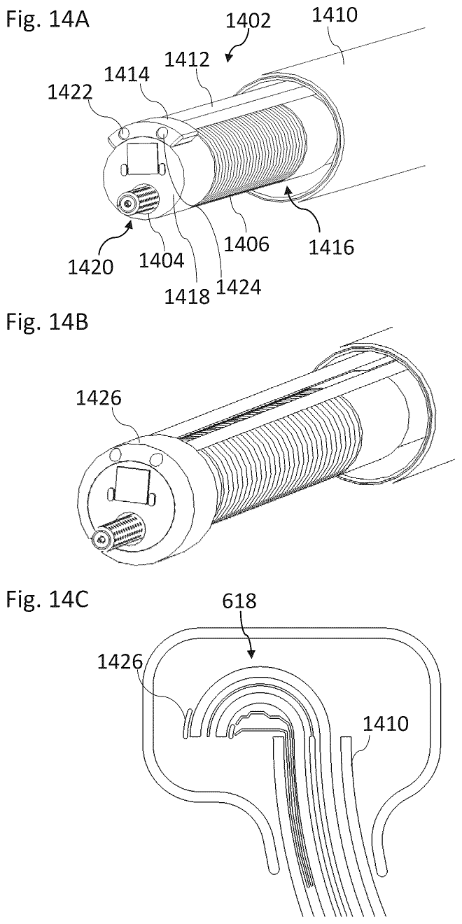

Reference is now made to FIGS. 13A-13D depicting a split connector of a working channel of an endoscope, according to some exemplary embodiments of the invention.

According to some exemplary embodiments, for example as shown in FIG. 13A, an endoscope 1302, for example an endoscope of a cryotherapy device, comprises an insertion tube 1304 coupled to a handle 1306. In some embodiments, the handle 1306 is shaped and sized to allow gripping of the handle body with a single hand, or two hands, while operating at least one button and/or switch of the handle 1306, for example to control the insertion and the steering of the insertion tube 1304 in a subject body.

According to some exemplary embodiments, the endoscope 1302 comprises at least one working channel 1308 within the insertion tube 1304, and optionally along at least 50%, for example at least 60%, at least 80%, at least 90% or any intermediate, smaller or larger percentage value, of an entire length of the insertion tube 1304. Optionally, the working channel 1308 passes within the handle 1306. In some embodiments, the working channel 1308 comprises at least one proximal opening, for example opening 1310, optionally positioned at a proximal end of the working channel. In some embodiments, the proximal opening 1310 comprises at least one valve 1312, for example a one-way valve. In some embodiments, the valve 1312 comprises a check valve, for example a duck bill check valve. Alternatively, a proximal opening of the working channel comprises a lock, without a valve.

According to some exemplary embodiments, for example as shown in FIGS. 13B and 13C, a split connector, for example split connector 1314, comprises a body 1316 which includes an inner lumen 1318. In some embodiments, the inner lumen comprises an outlet opening at a first end 1320 of the body 1316, and two or more inlet openings, for example openings 1322 and 1324, each at a different end of the body 1318. In some embodiments, the split connector is a Y connector. In some embodiments, the body 1318 comprises at least one bifurcation 1326, dividing the inner lumen 1318 into two spaced-apart lumens each terminating with an opening of openings 1322 and 1324.

According to some exemplary embodiments, for example as shown in FIG. 13D, a first end 1320 of the split connector 1314 is shaped and sized to penetrate into the opening 1310, optionally into valve 1312, for example into a duck bill valve of the working channel. In some embodiments, the first end 1320 of the split connector 1314 is shaped and sized to form a flow path between the working channel and the inner lumen 1318 of the split connector 1314. In some embodiments, insertion of the first end 1320, optionally comprising a plug, into the duck bill valve, locks the split connector 1314 to the opening 1310 of the working channel 1308. Optionally, the first end 1320 is a tapered end, shaped and sized to fit into the check valve 1312, for example into the duck bill check valve. Optionally, opening 1310 comprises a lock, for example a Luer lock to allow coupling of the split connector 1314 to the working channel proximal opening 1310.

According to some exemplary embodiments, at least one inlet opening of the split connector 1314 is connected to a tube 1311 that forms a fluid flow path 1313, for example a sealed flow path, via the split connector 1314 with the working channel. Optionally, the flow path 1313 is used to deliver pressure sensing flow into the working channel. In some embodiments, a different inlet opening of the split connector 1314 comprises a connector, for example a gasket 1326, which includes a seal 1328. In some embodiments, the seal 1326 comprises a leaf seal, configured to allow insertion of a cryocatheter 1330 through the gasket 1326 and the split connector 1314, into the working channel of the endoscope, without allowing exit of fluids through the connector 1326, for example fluids from the flow path 1313.

### Exemplary separated channels

According to some exemplary embodiments, a working channel of an endoscope is narrow, for example has a maximal inner width in a range of 1-6 mm, for example 1-5 mm, 2-5 mm, 2.5-5 mm or any intermediate, smaller or larger range of values. In some embodiments, a cryocatheter positioned within the narrow working channel include at least one washing fluid channel and at least one cryofluid channel, while at least one additional channel, optionally used for sensing is part of the endoscope, for example within one or more lumens in an overtube body, or is located within the evacuation channel. Optionally, the overtube comprises at least one inner lumen, for example a lumen for evacuation and/or a lumen for sensing. Reference is now made to FIGS. 14A-14C comprising at least one sensing channel of a cryotherapy device located outside the working channel, according to some exemplary embodiments of the invention.

According to some exemplary embodiments, a working channel of an endoscope is narrow, and allows insertion of a cryocatheter 1404 without sufficient space between the cryocatheter 1404 and a wall of the working channel for forming at least one additional channel, for example a sensing channel. In some embodiments, a cryotherapy device 1402 comprising the endoscope 1406 includes an overtube 1410 forming an evacuation channel between the overtube 1410 and the endoscope 1406. In some embodiments, at least one channel for example two or more channels, for example channels 1412 pass between the overtube 1410 and the endoscope 1406, optionally within the evacuation channel. In some embodiments, the channels 1412 comprise at least one pressure sensing channel and at least one temperature sensing channel. Alternatively, for example as shown in FIG. 10D, at least one sensing channel, for example two or more sensing channels, are integrated in the endoscope body, for example a single use endoscope.

According to some exemplary embodiments, at least one or both of the channels 1412 extend out from the overtube 1410 opening 1416, towards a distal tip 1418 of the endoscope 1406. In some embodiments, the cryotherapy device 1402 comprises at least one tip holder 1414 coupled to the endoscope, for example to the distal tip 1418, and configured to hold the channels 1412, for example close to the cryocatheter 1420. In some embodiments, holding one or both of the channels by the tip holder close to the cryocatheter, allows, for example, to position the channel openings, for example openings 1422 and 1424 close as possible to a target region within the hollow organ into which cryofluid is released by the cryocatheter, thereby optionally increasing the accuracy of the sensing.

According to some exemplary embodiments, for example as shown in FIG. 14A, the tip holder 1414 at least partly surrounds the endoscope distal tip 1418. Optionally, the tip holder is shaped as an arc. Alternatively, the tip holder, for example tip holder 1426 shown in FIG. 14B is shaped as a ring. Optionally, the holder, for example holder 1426 holds two or more channels at a distance from each other, for example to allow separation between the channels openings.

According to some exemplary embodiments, the at least one channel or the two or more channels that pass within overtube 1410, for example channels 1412 is formed from a tube. In some embodiments, for example as shown in FIG. 14C, a tube forming the channel, that is coupled to the tip holder 1426 is optionally loose, stretchable, and/or flexible, for example not to interfere with the bending of the steering section 618 of the endoscope.

According to some exemplary embodiments, for example as shown in FIG. 14D, at least one sensing channel 1432 located outside an endoscope 1434, optionally between an overtube 1436 and the endoscope 1434, is used to optionally introduce pressure flow and/or to sense pressure within the hollow organ. In some embodiments, the sensing channel 1432 is held, and optionally coupled to the endoscope 1434 by a holder, for example the holder 1426. In some embodiments, coupling the sensing channel 1432 to a tip of the endoscope 1434 places a distal opening 1438 close to a FOV 710, and or close to the target region 702 in the hollow organ into which washing fluid 1440 and cryofluid 1442 is released from the cryocatheter 1444.

### Exemplary Overtube

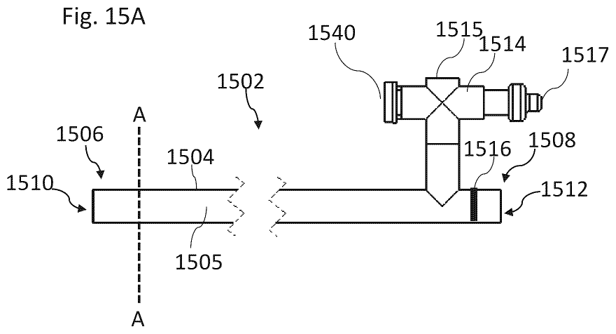

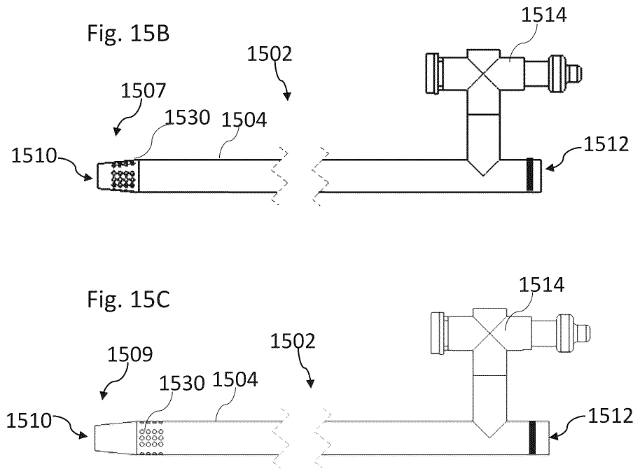

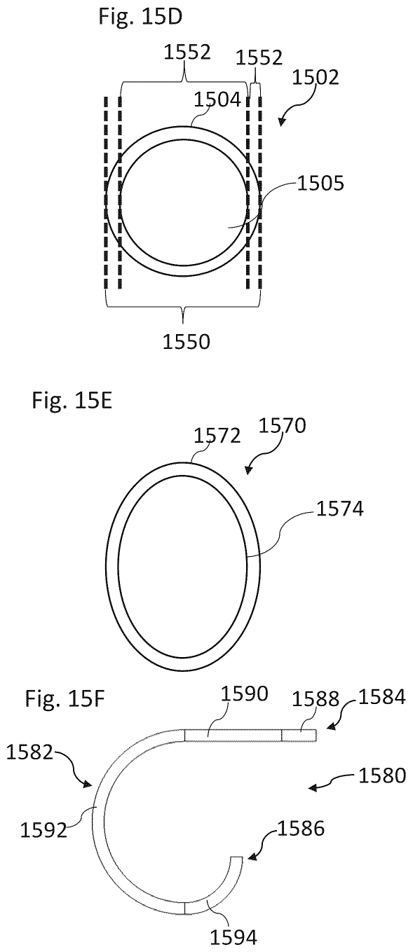

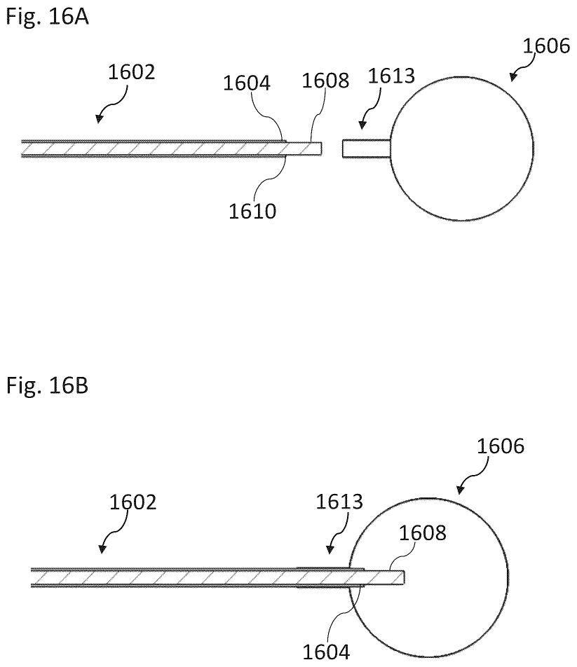

According to some exemplary embodiments, an overtube is shaped as a tube having a lumen, shaped and sized to receive an elongated surgical device, for example an endoscope or a cryocatheter, and to form a fluid flow path between the elongated surgical device and the overtube. Reference is now made to FIGS. 15A-15C, depicting overtube types, according to some exemplary embodiments of the invention.

According to some exemplary embodiments, an overtube, for example overtube 1502 comprises an elongated tubular body 1504 having an inner lumen 1505, a distal end 1506 and a proximal end 1508. In some embodiments, the inner lumen 1505 comprises at least one distal opening and at least one proximal opening 1512. In some embodiments, the overtube 1502 comprises at least one hollow flow regulator, for example flow regulator 1514 coupled to the elongated tubular body 1504, for example between the distal end 1506 and the proximal end 1508.

According to some exemplary embodiments, the inner lumen 1505 is shaped and sized to receive an elongated body of a surgical device, for example an endoscope or a cryocatheter, and to define a fluid flow path between the surgical device body and the overtube elongated tubular body. In some embodiments, the overtube 1502 comprises at least one seal, for example seal 1516, positioned in the inner lumen between said at least one proximal opening 1512 and the flow regulator 1514. In some embodiments, the seal 1516 is configured to seal the fluid flow path between the surgical device body and the overtube, for example to prevent escape of fluids within the fluid flow path through the proximal opening 1512. In some embodiments, the seal 1516 is optionally shaped as a ring, and comprises an opening configured to receive the surgical device elongated body, while sealing a gap between the surgical device elongated body and the overtube 1502.

According to some exemplary embodiments, for example as shown in FIG. 15B, a distal end of the overtube, for example distal end 1507 is tapered. In some embodiments, the tapered distal end is shaped and sized to penetrate into a hollow organ, or through a natural opening of the hollow organ, for example a urethra. In some embodiments, the overtube comprises one or more, for example a plurality of openings 1530 in a wall of the overtube at the tapered distal end 1507. In some embodiments, the openings are axially and/or circumferentially distributed in the overtube wall at the tapered distal end 1507. In some embodiments, the openings are inlet openings, positioned and/or distributed in the overtube wall, to allow, for example, efficient evacuation of fluids from the hollow organ into the overtube through the inlet openings.

According to some exemplary embodiments, for example as shown in FIG. 15C, the openings 1530 are axially and/or circumferentially distributed in a non-tapered region of the overtube wall, located proximally to the tapered distal end 1509.

Alternatively, the inlet openings are located in a wall of both the tapered end and in the non-tapered region of the overtube wall. In some embodiments, the inlet openings size is in a range of 0.1 mm-6 mm, for example 0.1 mm-2 mm, 1 mm-3 mm, 2 mm-5 mm or any intermediate, smaller or larger range of values.

According to some exemplary embodiments, the flow regulator 1514 is fluidically connected to the inner lumen 1505. Optionally, the flow regulator is integrated with the elongated tubular body 1504. In some embodiments, the flow regulator 1514 comprises at least one outlet opening, for example first outlet opening 1515 and second outlet opening 1517. In some embodiments, at least one opening of the first and second outlet opening 1515 and 1517, comprises a one-way valve, for example a check-valve, optionally configured to allow passive evacuation of fluids from the inner lumen 1505. Optionally, at least one opening of the first and second outlet openings comprises a connector or a lock, for example a Luer lock, configured to allow connection of a tube, for example a suction tube to the flow regulator 1514.

According to some exemplary embodiments, an outer diameter of the overtube 1502, for example an outer diameter of the body 1504 is up to 10 mm, for example up to 9 mm, up to 8 mm, up to 7 mm or any intermediate, smaller or larger value. According to some exemplary embodiments, the inner lumen 1505 is shaped and sized to receive an endoscope and/or a cryocatheter, for example an endoscope and/or a cryocatheter of a cryotherapy device. In some embodiments, a diameter of the lumen is up to 9 mm, for example up to 8 mm, up to 7 mm, up to 6 mm or any intermediate, smaller or larger value. According to some exemplary embodiments, for example as shown in FIGS. 11A-11E, the flow regulator comprises at least one movable flow path selector, for example a movable seal, configured to direct fluid flow from the inner lumen 1505 towards outlet opening 1515 and/or towards outlet opening 1517.

In some embodiments, the overtube comprises at least one user input interface, for example an activation button, configured to activate the cryofluid release and/or to select an evacuation flow path, for example an evacuation flow path through outlet opening 1515 and/or opening 1517.

Optionally, the flow regulator 1514 comprises at least one user input interface, for example activation button 1540 configured to activate the cryofluid release and/or to select an evacuation flow path, for example an evacuation flow path through outlet opening 1515 and/or opening 1517. In some embodiments, the activation button is functionally coupled to the movable flow path selector within the flow regulator 1514.

According to some exemplary embodiments, at least a portion of the body configured to be positioned within a subject body or within the hollow organ, is flexible and/or has a smooth outer surface, for example to prevent damage to tissue when penetrating into the hollow organ, or through a natural opening (e.g., a urethra).

Reference is now made to FIG. 15D, depicting a cross section AA of the elongated body of a flexible sheath, for example overtube 1502 shown in FIG. 15A, according to some exemplary embodiments of the invention.

According to some exemplary embodiments, the elongated body of the flexible sheath has large outer diameter, or an average diameter, for example an outer diameter in the range of 6 to 10 mm, and a wall thickness in a range between 0.05 mm and 0.3 mm, for example to allow a large inner width, for example a large inner diameter of the elongated body. In some embodiments, having an elongated body with a large outer diameter, or average diameter and a thin wall allows, for example to have a wide inner diameter of the sheath inner lumen, for example inner lumen 1505, suitable for passage of wide surgical tools and/or passage of large quantities of biological material, for example tissue, blood clots, debris and fluid. In some embodiments, for example as shown in FIG. 15E, an elongated body 1570 has a non-circular cross section, for example an oval, an ellipsoid or a polygon cross-section. In some embodiments, an outer surface 1572 or an inner surface 1574 of the body 1570 have a non-circular cross-section.

According to some exemplary embodiments, elongated body 1504 has a maximal outer width value, for example a maximal outer diameter value, in a range between 6 mm-10 mm, for example a maximal outer diameter value in a range between 6 mm-7 mm, 6.5 mm-7.5 mm, 6.5 mm-8 mm or any intermediate, smaller or larger range of values. In some embodiments, the elongated body cross section shape can be round, elliptic or other, while keeping large average outer diameter and a thin wall. In some embodiments, elongated body 1504 has a maximal inner width value, for example a maximal inner diameter in a range between 5 mm to 8.5 mm, for example a maximal inner diameter in a range between 5 mm to 6 mm, 5.5 mm to 6.5 mm, 6 mm to 7 mm, 6.5 mm to 7.5 mm or any intermediate, smaller or larger range of values. In some embodiments, a maximal thickness 1552 of a wall of the tubular body 1504 has a value in a range between 0.05 mm and 0.3 mm, for example in a range between 0.05 mm and 0.12 mm, in a range between 0.1 mm and 0.13 mm, in a range between 0.12 mm and 0.3 mm, in a range between 0.1 mm and 0.15 mm, or any intermediate, smaller or larger range of values.

According to some exemplary embodiments, the flexible sheath, for example at least a portion of the body 1504, is configured to bend in at least about 90 degrees, for example in at least about 95 degrees, in at least about 100 degrees, in at least about 110 degrees, or any intermediate, smaller or larger bending angle. In some embodiments, the flexible sheath, is formed from a metal tube that was cut, for example using laser, to generate patterned cuts in the body wall, to optionally allow bending of the body. Additionally or optionally, the body for example the outer surface of the body is coated with a coating, for example a scaling coating configured to seal the inner lumen of the body from the external environment outside the flexible sheath. In some embodiments, the coating comprises at least one of, a polymer, a Pebax® Jacket, for example dip coating of TPU or Silicon, a heat shrink for example a polyolefin heat shrink. Alternatively or additionally, the inner surface of the body is coated with the coating.

According to some exemplary embodiments, the body 1504 comprises a braided tube. Optionally, the body is formed from metal and/or carbon braiding, optionally coiled to allow reinforcement of the body and coated with polymer, for example Pebax® Jacket.

According to some exemplary embodiments, the sheath, or at least a portion of the body 1504, for example a distal portion of the body 1504 is flexible or has a flexible region, and/or is optionally formed from a flexible material. Alternatively or additionally, the sheath, or at least a portion of the body 1504, for example a distal portion of the body 1504 is configured to bend in at least 45 degrees, for example at least 90 degrees, at least 150 degrees or any intermediate, smaller or larger angle, relative to an unbent portion of the sheath or the body 1504, optionally without crimping or kinking. In some embodiments, at least a portion of the sheath or the sheath body is configured to bend in at least 45 degrees, relative to an unbent portion of the sheath while reducing an inner width of a cross-section of the bent portion in less than 10%, for example less than 5%, less than 3%, less than 1% or any intermediate, smaller or larger percentage value, relative to an inner width of the cross section when the bent portion is unbent, or straight.

According to some exemplary embodiments, for example as shown in FIG. 15F, a flexible sheath, for example flexible sheath 1580 comprises a body 1582 formed from two or more sections, each with a different bending limit. In some embodiments, a length of each section varies. In some embodiments, at least one section of the two or more sections is not coated, for example with an isolation, for example with a smoother coating or a pressure holding coating. In some embodiments, the body 1582, for example an elongated body, has a proximal end 1584 configured to be positioned outside the body of a subject, for example a human subject, and a distal end 1586, which is configured to be introduced into a hollow organ within the subject body. In some embodiments, the flexible sheath elongated body 1582 comprises two or more sections, for example a first section 1588 which is optionally a proximal section, a second section 1590, a third section 1592 which I optionally a main section of the body 1582, and a fourth section 1594, for example a distal section at the distal end 1586 of the body 1582. In some embodiments, each of the sections have a different length and/or a different bending limit.

According to some exemplary embodiments, the first section 1588, for example the proximal section, has a length within a range between 15 mm and 40 mm, for example in a range between 20 mm and 40 mm, in a range between 20 mm and 30 mm, in a range between 30 mm and 40 mm, or any intermediate, smaller or larger range of values. In some embodiments, the proximal section is non-bendable. In some embodiments, the proximal section optionally comprises at least one connector, configured to connect the proximal section of the body 1582 to a flow regulator or a check valve. Optionally, the proximal section is not coated with an insulation coating.

According to some exemplary embodiments, the second section 1590, has a length in a range between 30 mm and 90 mm, for example in a range between 40 mm and 90 mm, in a range between 40 mm and 70 mm, in a range between 40 mm and 60 mm or any intermediate, smaller or larger range of values. In some embodiments, the second section is bendable to a minimal radius of curvature of up to 100 mm, for example to a maximal radius of curvature of up to 200 mm, or any intermediate, smaller or larger value. In some embodiments, the second portion is rigid and/or non-bendable. Optionally, the second section is coated. According to some exemplary embodiments, the third section 1592, for example a main section of the flexible sheath has optionally a length which is larger than any other sections of the flexible sheath. In some embodiments, a length of the main section is in a range between 60 mm and 200 mm, for example in a range between 70 mm and 130 mm, in a range between 70 mm and 180 mm or any intermediate, smaller or larger range of values. In some embodiments, the main section is coated, for example with an isolating coating. In some embodiments, the coating smoothens the external surface of the sheath that is optionally placed in contact with tissue of the body. In some embodiments, the main section 1592 is bendable up to a radius of curvature in a range between 30 mm and 100 mm, for example 30 mm and 60 mm, 50 mm and 100 mm or any intermediate, smaller or larger range of values.

According to some exemplary embodiments, the fourth section 1594, for example the distal section of the flexible sheath has a length in a range between 20 mm and 60 mm, for example 20 mm and 40 mm, 30 mm and 50 mm, 40 mm and 60 mm, or any intermediate, smaller or larger range of values. In some embodiments, the distal section 1594 is bendable up to a radius of curvature in a range between 15 mm and 70 mm, for example up to a radius of curvature in a range between 15 mm and 40 mm, for example up to a radius of curvature in a range between 30 mm and 50 mm, or any intermediate, smaller or larger range of values.

According to some exemplary embodiments, the flexible sheath, for example overtube, is used alone or with an endoscope and/or at least one tool to perform procedures within a hollow organ, for example in the bladder. In some embodiments, the flexible sheath is used to remove objects, for example tissue, debris, blood clots, sediments, and stones, from the hollow organ. Alternatively or additionally, the flexible sheath is used to drain the hollow organ.

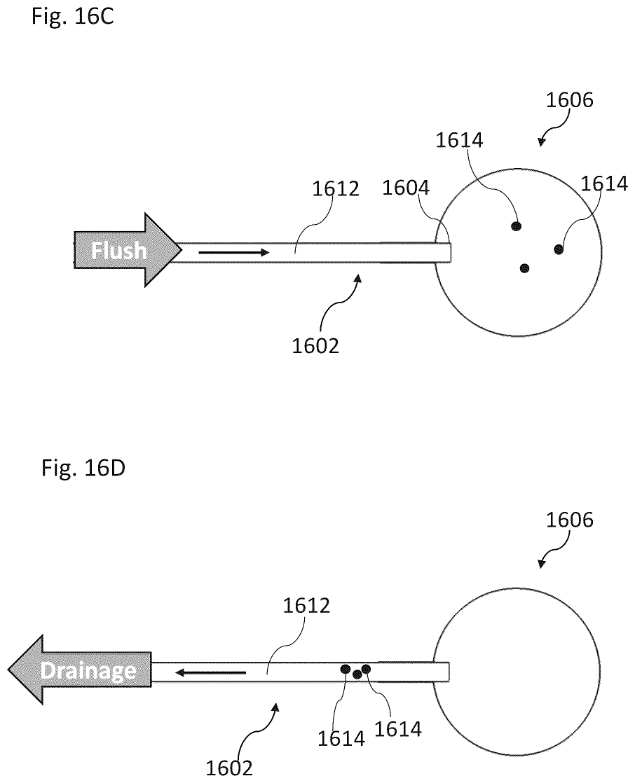

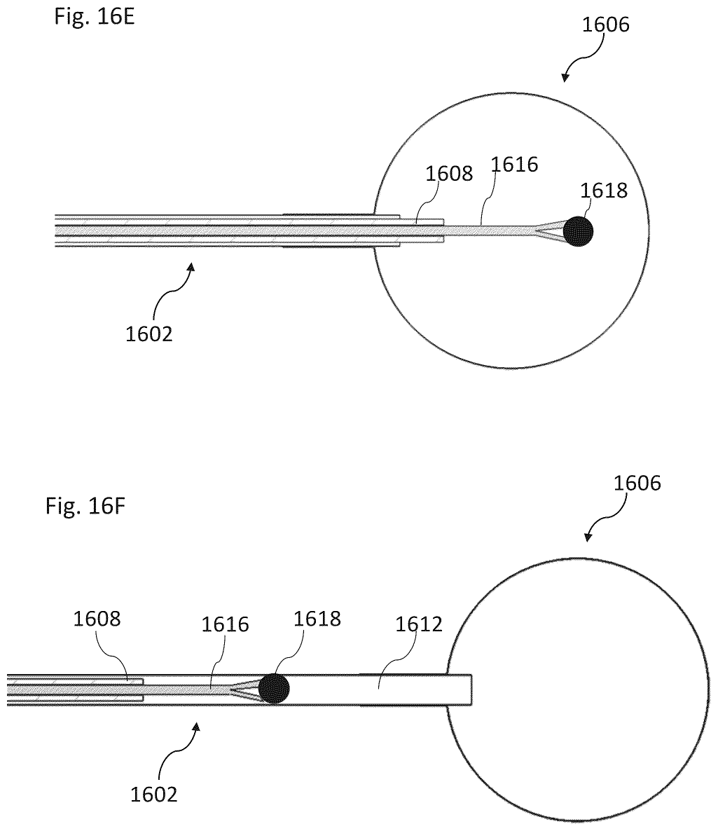

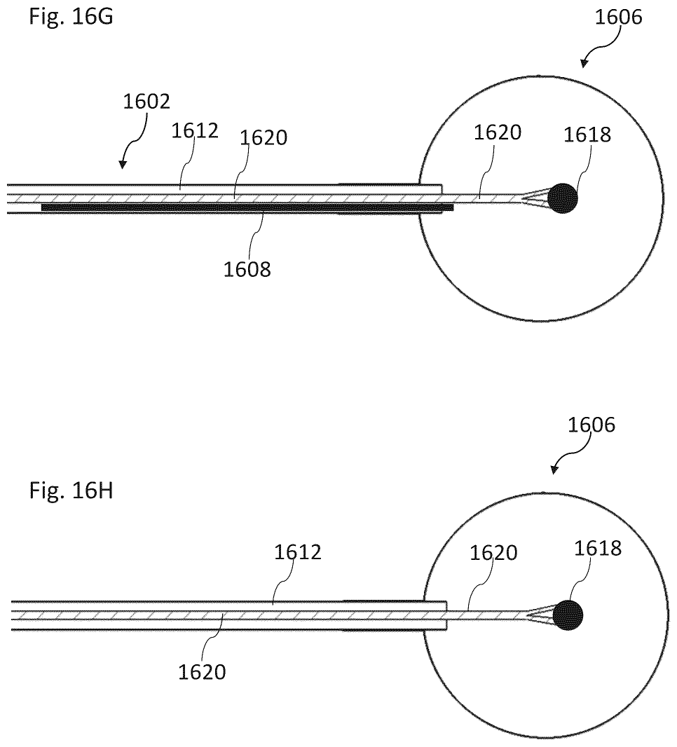

Reference is now made to FIGS. 16A and 16B, depicting navigation of a distal end of a flexible sheath into a hollow organ, according to some exemplary embodiments of the invention.

According to some exemplary embodiments, a flexible sheath, for example flexible sheath 1602 is introduced into a bony lumen, optionally under visualization. In some embodiments, the flexible sheath is advanced, optionally pushed, within natural openings of the body leading to the hollow organ, until a distal end 1604 of the flexible sheath which includes at least one distal opening of the flexible sheath inner lumen is placed within the hollow organ 1606.

According to some exemplary embodiments, for example as shown in FIG. 16A, a flexible sheath 1602 is advanced towards the hollow organ 1606 under visualization by an endoscope 1608 positioned within the flexible sheath inner lumen. In some embodiments, the endoscope 1608 extends at least partly from at least one distal opening 1610 of the flexible sheath inner lumen.

According to some exemplary embodiments, for example as shown in FIG. 16B, the distal end 1604 of the flexible sheath 1602 is introduced via an opening of the hollow organ into the hollow organ. In some embodiments, the hollow organ 1606 comprises a bladder and the flexible sheath distal end 1604 is introduced via the urethra 1613 into the bladder 1606, optionally under visualization by the endoscope 1608 or any other optical assembly positioned within the flexible sheath inner lumen that is configured to visualize a field of view located distally to the distal end of the flexible sheath.

According to some exemplary embodiments, once the distal end of the flexible sheath is positioned within the hollow organ 1606, for example within the bladder, the flexible sheath 1602 is used for flushing of the hollow organ. In some embodiments, the inner lumen 1612 of the flexible sheath is used to deliver fluid, for example washing fluid, into the hollow organ 1606, for example as shown in FIG. 16C. In some embodiments, flushing of the hollow organ 1606 allows, for example, to detach particles, for example particles 1614 from the inner surface of the hollow organ. In some embodiments, particles 1614 comprise at least one of tissue particles, debris, blood clots, and/or sediments, located within the hollow organ 1606. In some embodiments, the fluid, for example washing fluid, is delivered from a washing fluid source fluidically coupled to the flexible sheath inner lumen, for example via a flow regulator of the flexible sheath, for example flow regulator 1514 shown in FIGS. 15A-15C.

According to some exemplary embodiments, for example as shown in FIG. 16D, the flexible sheath 1602 is used to drain the hollow organ 1606, via the flexible sheath inner lumen 1612. In some embodiments, draining of the hollow organ 1606 allows, for example, to remove the particles 1614 from the hollow organ 1606, and optionally from the body, via the inner lumen 1612. In some embodiments, the hollow organ is drained by applying vacuum on the hollow organ via the flexible sheath inner lumen using a vacuum source coupled to the flexible sheath, for example to the flow regulator 1514 of the flexible sheath.

According to some exemplary embodiments, the flow regulator allows coupling of two or more fluid and/or vacuum sources using two or more openings of the flow regulator. In some embodiments, the flow regulator comprises a selector, for example to allow fluidically coupling of a single fluid or vacuum source to the inner lumen. In some embodiments, the selector allows to switch between one or more fluid sources and/or between one or more vacuum sources. Alternatively, the flexible sheath inner lumen is divided to separate channels, each is connected to a different source, for example a fluid source or a vacuum source. Optionally, each of the channels comprises at least one opening at the distal end of the flexible sheath.

According to some exemplary embodiments, for example as shown in FIGS. 16E and 16F, the flexible sheath 1602 allows to introduce one or more tools via a working channel of an endoscope 1608 located within the flexible sheath, into the hollow organ. In some embodiments, the one or more tools comprise a grasper, for example forceps 1616, configured to grasp large debris, for example a large stone, located within the hollow organ 1606. In some embodiments, the flexible sheath 1602 is used to remove the stone 1618 from the hollow organ 1606 by retracting the forceps 1616 and/or the endoscope 1608 towards a proximal end of the flexible sheath located outside the body, via the flexible sheath inner lumen 1612.

According to some exemplary embodiments, for example as shown in FIGS. 16G and 16H, the flexible sheath is used to introduce large tools, for example large surgical tool, that are too wide to be introduced into an endoscope's working channel, into the hollow organ in parallel to an endoscope or without an endoscope in the flexible sheath inner lumen. In some embodiments, for example as shown in FIG. 16G, a tool 1620 and endoscope 1608 are positioned within the inner lumen 1612 of the flexible sheath 1602. In some embodiments, the tool 1620 is positioned between the endoscope 1620 and the inner surface of the inner lumen 1612, and optionally in parallel to the endoscope 1620, within the inner lumen. In some embodiments, the inner lumen 1612 has an inner width, for example an inner diameter, in a range between 5 mm to 8 mm, which optionally allows to introduce tools having a maximal width between 1 mm and 5 mm, next to an endoscope, for example a cystoscope, for example a ureteroscope, and optionally in parallel to the endoscope.

According to some exemplary embodiments, for example as shown in FIG. 16H, a tool 1620 is introduced into the hollow organ 1606 via the flexible sheath inner lumen 1612, without an endoscope. Alternatively, the endoscope shown in FIG. 16G is removed from the flexible sheath inner lumen in order to allow capturing or grasping of a large particle, for example as shown in FIG. 16H and optionally removal of the particle from the hollow organ 1606 by retracting the tool within the flexible sheath inner lumen. In some embodiments, a large particle is a particle, for example a solid particle, having a minimal outer width of at least 3 mm, for example at least 3.5 mm, at least 4 mm, at least 4.5 mm, at least 5 mm, or any intermediate, smaller or larger value.

A potential advantage of having a flexible sheath with a wide inner lumen may be to allow insertion of large tools, for example large forceps into a hollow organ, for example into the bladder to remove large particles, for example stones or debris, that cannot be easily introduced within a working channel of an endoscope into the hollow organ.

It is expected that during the life of a patent maturing from this application many relevant endoscopes having at least one working channel will be developed; the scope of the terms endoscope, cystoscope, and working channel is intended to include all such new technologies a priori.

The terms “comprises”, “comprising”, “includes”, “including”, “has”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular forms “a”, “an” and “the” include plural references unless the context clearly dictates otherwise.

Throughout this application, embodiments of this invention may be presented with reference to a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as “from 1 to 6” should be considered to have specifically disclosed subranges such as “from 1 to 3”, “from 1 to 4”, “from 1 to 5”, “from 2 to 4”, “from 2 to 6”, “from 3 to 6”, etc.; as well as individual numbers within that range, for example, 1, 2, 3, 4, 5, and 6. This applies regardless of the breadth of the range.

Whenever a numerical range is indicated herein (for example “10-15”, “10 to 15”, or any pair of numbers linked by these another such range indication), it is meant to include any number (fractional or integral) within the indicated range limits, including the range limits, unless the context clearly dictates otherwise. The phrases “range/ranging/ranges between” a first indicate number and a second indicate number and “range/ranging/ranges from” a first indicate number “to”, “up to”, “until” or “through” (or another such range-indicating term) a second indicate number are used herein interchangeably and are meant to include the first and second indicated numbers and all the fractional and integral numbers therebetween

Unless otherwise indicated, numbers used herein and any number ranges based thereon are approximations within the accuracy of reasonable measurement and rounding errors as understood by persons skilled in the art

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1-50. (canceled)

51. A cryocatheter, comprising:
an elongated body terminating with a bendable distal section, which is shaped and sized to penetrate into a hollow organ, wherein said bendable distal section comprises a distal tip;
at least one cryofluid channel located within said bendable distal section, comprises at least one distal opening at said distal tip configured to release cryofluid from said at least one cryofluid channel;
at least one washing fluid channel located within said bendable distal section and coupled to said at least one cryofluid channel, wherein said at least one washing fluid channel comprises at least one distal opening at said distal tip configured to release washing fluid from said at least one washing channel;
wherein bending of said bendable distal section in an angle of at least 45 degrees relative to a section of the elongated body proximal to said bendable distal section, reduces cryofluid flow through said distal opening by less than 10 percent relative to cryofluid flow through said distal opening when said bendable distal section is unbent.

52. A cryocatheter according to claim 51, wherein a bending radius of said bendable distal section is in a range of 5-20 mm.

53. A cryocatheter according to claim 51, wherein said at least one cryofluid channel in said elongated body is formed from an elongated cryofluid tube comprising a distal flexible section at said bendable distal section, and a proximal section, wherein an outer diameter of said distal flexible section is smaller than an outer diameter of said proximal section to allow bending of said distal flexible section in an angle of at least 45 degrees relative to said proximal section.

54. A cryocatheter according to claim 53, wherein said flexible distal section of said at least one cryofluid channel is configured to bend in a radius of curvature which is smaller than a radius of curvature of said proximal section.

55. A cryocatheter according to claim 53, wherein a length of said distal flexible section of said at least one cryofluid channel is in a range of 20-150 mm.

56. A cryocatheter according to claim 53, wherein an outer diameter of said distal flexible section of said at least one cryofluid channel is in a range of 0.1-2 mm.

57. A cryocatheter according to claim 53, wherein a wall of said distal flexible section has a width in a range of 0.02 mm to 0.2 mm.

58. A cryocatheter according to claim 53, wherein said distal flexible section of said at least one cryofluid channel is formed from Nitinol, Titanium, or Stainless-Steel.

59. A cryocatheter according to claim 51, wherein said bendable distal section of said elongated body is configured to bend in at least 45 degrees without crimping or kinking.

60. A cryocatheter according to claim 51, wherein said at least one cryofluid channel is coaxially positioned within said at least one washing fluid channel.

61. A cryocatheter according to claim 51, wherein said cryocatheter is shaped and sized to be positioned within a working channel of a flexible endoscope and to extend out from a distal opening of the working channel.

62. A cryocatheter according to claim 51, wherein said elongated body has an outer diameter in a range of 1 mm to 3 mm.

63. A cryocatheter according to claim 51, wherein said at least one washing fluid channel comprises a plurality of openings in the wall of the at least one washing fluid channel distributed on a circumference of the washing fluid channel.

64. A cryotherapy device, comprising:
an endoscope comprising:
an elongated insertion tube having a distal tip shaped and sized to penetrate into a hollow organ;
a working channel within said elongated insertion tube comprising a proximal opening and a distal opening at said distal tip;
a cryocatheter according to claim 51, wherein said cryocatheter is shaped and sized to move within the working channel and to extend at least partly from said working channel distal opening, to position said at least one washing fluid distal opening in a selected distance and/or orientation relative to said elongated insertion tube distal tip.

65. A device according to claim 64, wherein said endoscope comprises at least one optical element at said distal tip, and wherein said cryocatheter controllably moves within said working channel to position said at least one washing fluid distal opening in a selected distance and/or orientation relative to said optical element.

66. A device according to claim 64, wherein said endoscope comprises at least one optical element at said distal tip defining a field of view (FOV) distal to said at least one optical element and wherein said cryocatheter controllably moves within said working channel to position said at least one washing fluid distal opening in a selected distance and/or orientation relative to said FOV.

67. A cryotherapy method, comprising:
navigating a cryotherapy device comprising an endoscope and a cryocatheter into a hollow organ, wherein said cryocatheter comprises at least one cryofluid channel and at least one washing fluid channel coupled to each other;
bending a bendable distal section of said endoscope positioned within said hollow organ in at least 45 degrees relative to a section of said endoscope located outside said hollow organ;
releasing cryofluid from at least one distal opening of said at least one cryofluid channel located at said bendable distal section, when said bendable distal section is bent.

68. A method according to claim 67, comprising:
releasing washing fluid from a least one washing fluid opening of said at least one washing fluid channel located at said bendable distal section, when said bendable distal section is bent, wherein said washing fluid is released before, during and/or after the cryofluid release.

69. A method according to claim 67, wherein bending of said endoscope bendable distal section reduces flow of said cryofluid from said distal opening during said cryofluid releasing by less than 10 percent relative to cryofluid flow through said distal opening when said endoscope bendable distal section is unbent.

70. A method according to claim 67, wherein said navigating, said bending and said releasing is performed during a treatment for ablating tissue within said hollow organ.
